SEQUENCE LISTING (39 sequences)

SEQ ID NO: 1 - DNA, 19 bp, Artificial Sequence
  forward primer for 8346m3 locus
cgacaaagattggagaccc

SEQ ID NO: 2 - DNA, 20 bp, Artificial Sequence
  reverse primer for 8346m3 locus
tttcaatgcagtgtgattcc

SEQ ID NO: 3 - DNA, 18 bp, Artificial Sequence
  forward primer for 7961m4 locus
aagcatcaccaaaacggc

SEQ ID NO: 4 - DNA, 18 bp, Artificial Sequence
  reverse primer for 7961m4 locus
ttacatttgcgtctcggc

SEQ ID NO: 5 - DNA, 18 bp, Artificial Sequence
  forward primer for 11745m3 locus
ttctgttcgcgtgttccc

SEQ ID NO: 6 - DNA, 18 bp, Artificial Sequence
  reverse primer for 11745m3 locus
tgttctgccactggaggg

SEQ ID NO: 7 - DNA, 19 bp, Artificial Sequence
  forward primer for 12406 locus
gctgaagttgtctccgtgc

SEQ ID NO: 8 - DNA, 20 bp, Artificial Sequence
  reverse primer for 12406 locus
ttttcaggcatatcaggagc

SEQ ID NO: 9 - DNA, 20 bp, Artificial Sequence
  forward primer for 11543m5 locus
ttctgacacagccatgaacc

SEQ ID NO: 10 - DNA, 19 bp, Artificial Sequence
  reverse primer for 11543m5 locus
cccctttccaaaattcacc

SEQ ID NO: 11 - DNA, 20 bp, Artificial Sequence
  forward primer for 530m4 locus
gttcacaggagtgttatgcc

SEQ ID NO: 12 - DNA, 19 bp, Artificial Sequence
  reverse primer for 530m4 locus
tctcatttgcacgttctcc

SEQ ID NO: 13 - DNA, 20 bp, Artificial Sequence
  forward primer for 11401m4 locus
tgcagacagaaccgagaagg

SEQ ID NO: 14 - DNA, 18 bp, Artificial Sequence
  reverse primer for 11401m4 locus
tgggccacgattcttacg

SEQ ID NO: 15 - DNA, 18 bp, Artificial Sequence
  forward primer for 441m6 locus
gccttccggaactatcgc

SEQ ID NO: 16 - DNA, 18 bp, Artificial Sequence
  reverse primer for 441m6 locus
tgcgaagatggtgtcacg

SEQ ID NO: 17 - DNA, 17 bp, Artificial Sequence
  forward primer for 11292m4 locus
tgcgaatggagctctgg

SEQ ID NO: 18 - DNA, 19 bp, Artificial Sequence
  reverse primer for 11292m4 locus
tcatttcgtccattcatgc

SEQ ID NO: 19 - DNA, 18 bp, Artificial Sequence
  forward primer for 8499m4 locus
aaaccgtgggttaagggc

SEQ ID NO: 20 - DNA, 18 bp, Artificial Sequence
  reverse primer for 8499m4 locus
cgatggaattattcgcgg

SEQ ID NO: 21 - DNA, 18 bp, Artificial Sequence
  forward primer for 7203m5 locus
atttcctcaccattcccc

SEQ ID NO: 22 - DNA, 19 bp, Artificial Sequence
  reverse primer for 7203m5 locus
tgagggaaaacaacactcc

SEQ ID NO: 23 - DNA, 18 bp, Artificial Sequence
  forward primer for 10366m5 locus
caacgactgaaaggcagc

SEQ ID NO: 24 - DNA, 20 bp, Artificial Sequence
  reverse primer for 10366m5 locus
ggctttcgacttttatgtcc

SEQ ID NO: 25 - DNA, 18 bp, Artificial Sequence
  forward primer for 12130m5 locus
tgagggtaaaggcggacc

SEQ ID NO: 26 - DNA, 19 bp, Artificial Sequence
  reverse primer for 12130m5 locus
ttttgctttatccgcatcg

SEQ ID NO: 27 - DNA, 20 bp, Artificial Sequence
  forward primer for 4546m2 locus
tgtgcaatgaaaatttcccc

SEQ ID NO: 28 - DNA, 19 bp, Artificial Sequence
  reverse primer for 4546m2 locus
cagttcccttgttcctggg

SEQ ID NO: 29 - DNA, 19 bp, Artificial Sequence
  forward primer for 9079m3 locus
tttcgtgttatagctcccg

SEQ ID NO: 30 - DNA, 19 bp, Artificial Sequence
  reverse primer for 9079m3 locus
cctggcttttaatctgagg

SEQ ID NO: 31 - DNA, 17 bp, Artificial Sequence
  forward primer for 11192m4 locus
tgaggaccctcccttgc

SEQ ID NO: 32 - DNA, 18 bp, Artificial Sequence
  reverse primer for 11192m4 locus
aggctgcatctggtttcc

SEQ ID NO: 33 - DNA, 18 bp, Artificial Sequence
  forward primer for 8010m6 locus
acgctgtggtaaagcacg

SEQ ID NO: 34 - DNA, 18 bp, Artificial Sequence
  reverse primer for 8010m6 locus
cacttgacaccacgctgc

SEQ ID NO: 35 - DNA, 19 bp, Artificial Sequence
  forward primer for 12198m3 locus
catctccaaggaactttgc

SEQ ID NO: 36 - DNA, 18 bp, Artificial Sequence
  reverse primer for 12198m3 locus
ttcacgttgtgttttggc

SEQ ID NO: 37 - DNA, 18 bp, Artificial Sequence
  forward primer for 7850m4 locus
atgcctgcaagtgtttgg

SEQ ID NO: 38 - DNA, 25 bp, Artificial Sequence
  reverse primer for 7850m4 locus
gtttctttaacgtcacgcgttgtcc

SEQ ID NO: 39 - DNA, 17 bp, Artificial Sequence
  M13R primer
caggaaacagctatgac

(19) United States
(12) Patent Application Publication
(10) Pub. No.: US 2017/0058363 A1
(43) Pub. Date: Mar. 2, 2017

(54) CROSS-SPECIES AND INTRASPECIES MICROSATELLITE MARKERS FOR ACROPORA CORALS

(71) Applicant: OKINAWA INSTITUTE OF SCIENCE AND TECHNOLOGY SCHOOL CORPORATION, Okinawa (JP)

(72) Inventors: Chuya Shinzato, Okinawa (JP); Sutada Mungpakdee, Okinawa (JP); Yuichi Nakajima, Okinawa (JP)

(21) Appl. No.: 15/303,416

(22) PCT Filed: Apr. 10, 2015
(86) PCT No.: PCT/JP2015/061770

§ 371 Date: Oct. 11, 2016

Related U.S. Application Data
(60) Provisional application No. 61/977,797, filed on Apr. 10, 2014.

(30) Foreign Application Priority Data
Apr. 28, 2014 (JP) 2014-093062

(51) Int. Cl. C12Q 1/68

(52) U.S. Cl. CPC C12Q 1/6888; C12Q 2600/156; C12Q 2600/16

(57) ABSTRACT

A method for identifying a subject of Acropora genus to an Acropora species comprises of detecting one or more microsatellite loci in the genome of the subject, wherein the microsatellite loci are selected from the group consisting of a microsatellite locus 8346m3 and others. A method for quantifying an index of level of chimerism of a coral colony restored by transplanting an Acropora first coral colony of an Acropora species into a second coral colony in need of restoration that belongs to the same species as the first coral colony comprises of identifying one or more microsatellite loci whose PCR fragment size, amplified with genomic DNA derived from the first coral colony as template, is different from the PCR fragment size amplified with genomic DNA derived from the second coral colony.

### FIELD OF THE INVENTION

The present invention relates to microsatellite markers for Acropora genus and, in particular, microsatellite markers universal to Acropora genus.

### BACKGROUND OF THE INVENTION

### Introduction

Acropora (Scleractinia, Acroporidae) is a common, emblematic genus of reef-building corals. It is also one of the most widespread coral genera, ranging from the Red Sea through the Indo-Pacific Ocean to the Caribbean, and has the largest number of extant species (113) (Wallace, 1999). The fossil record suggests that the genus probably originated about 50 million years ago (MYR) (Veron, 1995). There are two distinct groups of Acropora corals: mass spawning acroporids and “early spawners,” that spawn 1.5-3 h earlier than other mass-spawning species (Hatta et al., 1999; Fukami et al., 2000). The two groups are believed to have diverged 6.6 MYA (Fukami et al., 2000). Major diversification within these groups has occurred more recently (2 MYR) (Veron, 1995). In addition, molecular phylogenetic analyses using a single copy gene PaxC intron and mitochondrial markers show that Acropora corals can be divided into four major clades (van Oppen et al., 2001; Marquez et al., 2002) (FIG. 1). Early spawning species, including A. tenuis, belong to the most basal Glade (Clade I) (Fukami et al., 2000; van Oppen et al., 2001), while most of mass-spawning species belong to Clade III. Clade IV has relatively small number of species, including A. digitifera (van Oppen et al., 2001).

Coral reefs are estimated to harbor around one-third of all described marine species (Knowlton et al., 2010); however, they face a range of anthropogenic challenges, including ocean acidification and increasing seawater temperatures (e.g., Hoegh-Guldberg et al., 2007). Although Acropora species are major components of coral reefs worldwide, they are the most sensitive to increased water temperatures (Loya et al., 2001) and are expected to decline in the near future (Alvarez-Filip et al., 2013). For proper maintenance and conservation of Acropora corals, it is important to understand genetic diversity and connectivity among populations. High-resolution genetic markers, such as microsatellites, are essential for such studies. Previous studies have succeeded in developing microsatellite markers specific to several Acropora species, e.g. A. palmata (Baums et al., 2005), A. millepora (Van Oppen et al., 2007), A. cytherea (Concepcion et al., 2010), and Acropora sp1 and A. digitifera (Nakajima et al., 2009). However, cross-species amplification was confirmed for several markers (Nakajima et al., 2009). Because microsatellite markers are currently available for only about 5 of the 113 Acropora species, an increased number of “universal” Acropora microsatellite markers would be extremely useful. In this study we developed cross-species microsatellite markers that can be applied to a variety of Acropora species. To achieve this we used two Acropora species that belong to taxonomically distant clades (I: A. tenuis, IV: A. digitifera) and we took advantage of next-generation sequencing technology to design novel microsatellite primer pairs that can be used for both species.

### SUMMARY OF THE INVENTION

In one aspect of the present invention, there is provided a method for identifying a subject to Acropora genus, comprising the steps of:

detecting one or more microsatellite loci in the genome of the subject, wherein said microsatellite loci are listed in Table 1 or are selected from the group consisting of:
(i) a microsatellite locus 8346m3 wherein its 5′ flanking region comprises a nucleotide sequence of SEQ ID NO:1 and its 3′ flanking region comprises a nucleotide sequence complementary to SEQ ID NO:2;
(ii) a microsatellite locus 7961m4 wherein its 5′ flanking region comprises a nucleotide sequence of SEQ ID NO:3 and its 3′ flanking region comprises a nucleotide sequence complementary to SEQ ID NO:4;
(iii) a microsatellite locus 11745m3 wherein its 5′ flanking region comprises a nucleotide sequence of SEQ ID NO:5 and its 3′ flanking region comprises a nucleotide sequence complementary to SEQ ID NO:6;
(iv) a microsatellite locus 12406m3 wherein its 5′ flanking region comprises a nucleotide sequence of SEQ ID NO:7 and its 3′ flanking region comprises a nucleotide sequence complementary to SEQ ID NO:8;
(v) a microsatellite locus 11543m5 wherein its 5′ flanking region comprises a nucleotide sequence of SEQ ID NO:9 and its 3′ flanking region comprises a nucleotide sequence complementary to SEQ ID NO:10;
(vi) a microsatellite locus 530m4 wherein its 5′ flanking region comprises a nucleotide sequence of SEQ ID NO:11 and its 3′ flanking region comprises a nucleotide sequence complementary to SEQ ID NO:12;
(vii) a microsatellite locus 11401m4 wherein its 5′ flanking region comprises a nucleotide sequence of SEQ ID NO:13 and its 3′ flanking region comprises a nucleotide sequence complementary to SEQ ID NO:14;
(viii) a microsatellite locus 441m6 wherein its 5′ flanking region comprises a nucleotide sequence of SEQ ID NO:15 and its 3′ flanking region comprises a nucleotide sequence complementary to SEQ ID NO:16;
(ix) a microsatellite locus 11292m4 wherein its 5′ flanking region comprises a nucleotide sequence of SEQ ID NO:17 and its 3′ flanking region comprises a nucleotide sequence complementary to SEQ ID NO:18;
(x) a microsatellite locus 8499m4 wherein its 5′ flanking region comprises a nucleotide sequence of SEQ ID NO:19 and its 3′ flanking region comprises a nucleotide sequence complementary to SEQ ID NO:20;
(xi) a microsatellite locus 7203m5 wherein its 5′ flanking region comprises a nucleotide sequence of SEQ ID NO:21 and its 3′ flanking region comprises a nucleotide sequence complementary to SEQ ID NO:22;
(xii) a microsatellite locus 10366m5 wherein its 5′ flanking region comprises a nucleotide sequence of SEQ ID NO:23 and its 3′ flanking region comprises a nucleotide sequence complementary to SEQ ID NO:24;
(xiii) a microsatellite locus 12130m5 wherein its 5′ flanking region comprises a nucleotide sequence of SEQ ID NO:25 and its 3′ flanking region comprises a nucleotide sequence complementary to SEQ ID NO:26;
(xiv) a microsatellite locus 4546m2 wherein its 5′ flanking region comprises a nucleotide sequence of SEQ ID NO:27 and its 3′ flanking region comprises a nucleotide sequence complementary to SEQ ID NO:28;
(xv) a microsatellite locus 9079m3 wherein its 5′ flanking region comprises a nucleotide sequence of SEQ ID NO:29 and its 3′ flanking region comprises a nucleotide sequence complementary to SEQ ID NO:30;
(xvi) a microsatellite locus 11192m4 wherein its 5′ flanking region comprises a nucleotide sequence of SEQ ID NO:31 and its 3′ flanking region comprises a nucleotide sequence complementary to SEQ ID NO:32;
(xvii) a microsatellite locus 8010m6 wherein its 5′ flanking region comprises a nucleotide sequence of SEQ ID NO:33 and its 3′ flanking region comprises a nucleotide sequence complementary to SEQ ID NO:34;
(xviii) a microsatellite locus 12198m3 wherein its 5′ flanking region comprises a nucleotide sequence of SEQ ID NO:35 and its 3′ flanking region comprises a nucleotide sequence complementary to SEQ ID NO:36; and
(xix) a microsatellite locus 7850m4 wherein its 5′ flanking region comprises a nucleotide sequence of SEQ ID NO:37 and its 3′ flanking region comprises a nucleotide sequence complementary to SEQ ID NO:38.

In the method for identifying a subject to Acropora genus, the detection of a microsatellite locus in the genome of the subject may be performed by using one or more primer pairs selected from the group consisting of:

(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38.

In the method for identifying a subject to Acropora genus, the detection of a microsatellite locus in the genome of the subject may be performed by using one or more primer pairs selected from the group consisting of:

(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2, wherein the primers amplify DNA of the microsatellite locus 8346m3 using genomic DNA of the subject as template;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4, wherein the primers amplify DNA of the microsatellite locus 7961m4 using genomic DNA of the subject as template;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6, wherein the primers amplify DNA of the microsatellite locus 11745m3 using genomic DNA of the subject as template;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8, wherein the primers amplify DNA of the microsatellite locus 12406m3 using genomic DNA of the subject as template;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10, wherein the primers amplify DNA of the microsatellite locus 11543m5 using genomic DNA of the subject as template;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12, wherein the primers amplify DNA of the microsatellite locus 530m4 using genomic DNA of the subject as template;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14, wherein the primers amplify DNA of the microsatellite locus 11401m4 using genomic DNA of the subject as template;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16, wherein the primers amplify DNA of the microsatellite locus 441m6 using genomic DNA of the subject as template;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18, wherein the primers amplify DNA of the microsatellite locus 11292m4 using genomic DNA of the subject as template;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20, wherein the primers amplify DNA of the microsatellite locus 8499m4 using genomic DNA of the subject as template;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22, wherein the primers amplify DNA of the microsatellite locus 7203m5 using genomic DNA of the subject as template;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26, wherein the primers amplify DNA of the microsatellite locus 12130m5 using genomic DNA of the subject as template;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28, wherein the primers amplify DNA of the microsatellite locus 4546m2 using genomic DNA of the subject as template;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30, wherein the primers amplify DNA of the microsatellite locus 9079m3 using genomic DNA of the subject as template;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32, wherein the primers amplify DNA of the microsatellite locus 11192m4 using genomic DNA of the subject as template;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34, wherein the primers amplify DNA of the microsatellite locus 8010m6 using genomic DNA of the subject as template;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36, wherein the primers amplify DNA of the microsatellite locus 12198m3 using genomic DNA of the subject as template; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38, wherein the primers amplify DNA of the microsatellite locus 7850m4 using genomic DNA of the subject as template.

In the method for identifying a subject to Acropora genus, the one or more primer pairs may be selected from the group consisting of:

(i) a primer comprising a nucleotide sequence of SEQ ID NO:1 and a primer comprising a nucleotide sequence of SEQ ID NO:2:
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6.
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:16;
(viii) a primer comprising a nucleotide sequence of SEQ ID NO:15 and a primer comprising a nucleotide sequence of SEQ ID NO:16;
(ix) a primer comprising a nucleotide sequence of SEQ NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer comprising a nucleotide sequence of SEQ ID NO:36; and
(xix) a primer comprising a nucleotide sequence of SEQ ID NO:37 and a primer comprising a nucleotide sequence of SEQ ID NO:38.

In the method for identifying a subject to Acropora genus, one of the primer pair may further comprise a nucleotide sequence of SEQ ID NO:39 (M13 primer) at its 5′ end.

In another aspect of the present invention, there is provided a primer pair for amplifying a microsatellite locus in the genome of Acropora genus, wherein said microsatellite locus is selected from Table 1 or is selected from the group consisting of:

(i) a microsatellite locus 8346m3 wherein its 5′ flanking region comprises a nucleotide sequence of SEQ ID NO:1 and its 3′ flanking region comprises a nucleotide sequence complementary to SEQ ID NO:2;
(ii) a microsatellite locus 7961m4 wherein its 5′ flanking region comprises a nucleotide sequence of SEQ ID NO:3 and its 3′ flanking region comprises a nucleotide sequence complementary to SEQ ID NO:4;
(iii) a microsatellite locus 11745m3 wherein its 5′ flanking region comprises a nucleotide sequence of SEQ ID NO:5 and its 3′ flanking region comprises a nucleotide sequence complementary to SEQ ID NO:6;
(iv) a microsatellite locus 12406m3 wherein its 5′ flanking region comprises a nucleotide sequence of SEQ ID NO:7 and its 3′ flanking region comprises a nucleotide sequence complementary to SEQ ID NO:8;
(v) a microsatellite locus 11543m5 wherein its 5′ flanking region comprises a nucleotide sequence of SEQ ID NO:9 and its 3′ flanking region comprises a nucleotide sequence complementary to SEQ ID NO:10;
(vi) a microsatellite locus 530m4 wherein its 5′ flanking region comprises a nucleotide sequence of SEQ ID NO:11 and its 3′ flanking region comprises a nucleotide sequence complementary to SEQ ID NO:12;
(vii) a microsatellite locus 11401m4 wherein its 5′ flanking region comprises a nucleotide sequence of SEQ ID NO:13 and its 3′ flanking region comprises a nucleotide sequence complementary to SEQ ID NO:14;
(viii) a microsatellite locus 441m6 wherein its 5′ flanking region comprises a nucleotide sequence of SEQ ID NO:15 and its 3′ flanking region comprises a nucleotide sequence complementary to SEQ ID NO:16;
(ix) a microsatellite locus 11292m4 wherein its 5′ flanking region comprises a nucleotide sequence of SEQ ID NO:17 and its 3′ flanking region comprises a nucleotide sequence complementary to SEQ ID NO:18;
(x) a microsatellite locus 8499m4 wherein its 5′ flanking region comprises a nucleotide sequence of SEQ ID NO:19 and its 3′ flanking region comprises a nucleotide sequence complementary to SEQ ID NO:20;
(xi) a microsatellite locus 7203m5 wherein its 5′ flanking region comprises a nucleotide sequence of SEQ ID NO:21 and its 3′ flanking region comprises a nucleotide sequence complementary to SEQ ID NO:22;
(xii) a microsatellite locus 10366m5 wherein its 5′ flanking region comprises a nucleotide sequence of SEQ ID NO:23 and its 3′ flanking region comprises a nucleotide sequence complementary to SEQ ID NO:24;
(xiii) a microsatellite locus 12130m5 wherein its 5′ flanking region comprises a nucleotide sequence of SEQ ID NO:25 and its 3′ flanking region comprises a nucleotide sequence complementary to SEQ ID NO:26;
(xiv) a microsatellite locus 4546m2 wherein its 5′ flanking region comprises a nucleotide sequence of SEQ ID NO:27 and its 3′ flanking region comprises a nucleotide sequence complementary to SEQ ID NO:28;
(xv) a microsatellite locus 9079m3 wherein its 5′ flanking region comprises a nucleotide sequence of SEQ ID NO:29 and its 3′ flanking region comprises a nucleotide sequence complementary to SEQ ID NO:30;
(xvi) a microsatellite locus 11192m4 wherein its 5′ flanking region comprises a nucleotide sequence of SEQ ID NO:31 and its 3′ flanking region comprises a nucleotide sequence complementary to SEQ ID NO:32;
(xvii) a microsatellite locus 8010m6 wherein its 5′ flanking region comprises a nucleotide sequence of SEQ ID NO:33 and its 3′ flanking region comprises a nucleotide sequence complementary to SEQ ID NO:34;
(xviii) a microsatellite locus 12198m3 wherein its 5′ flanking region comprises a nucleotide sequence of SEQ ID NO:35 and its 3′ flanking region comprises a nucleotide sequence complementary to SEQ ID NO:36; and
(xix) a microsatellite locus 7850m4 wherein its 5′ flanking region comprises a nucleotide sequence of SEQ ID NO:37 and its 3′ flanking region comprises a nucleotide sequence complementary to SEQ ID NO:38.

In the primer pair for amplifying a microsatellite locus in the genome of Acropora genus, the primer pair may be selected from the group consisting of:

(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38.

In the primer pair for amplifying a microsatellite locus in the genome of Acropora genus, the primer pair may be selected from the group consisting of:

(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2, wherein the primers amplify DNA of the microsatellite locus 8346m3 using genomic DNA of the subject as template;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4, wherein the primers amplify DNA of the microsatellite locus 7961m4 using genomic DNA of the subject as template;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6, wherein the primers amplify DNA of the microsatellite locus 11745m3 using genomic DNA of the subject as template;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8, wherein the primers amplify DNA of the microsatellite locus 12406m3 using genomic DNA of the subject as template;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10, wherein the primers amplify DNA of the microsatellite locus 11543m5 using genomic DNA of the subject as template;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12, wherein the primers amplify DNA of the microsatellite locus 530m4 using genomic DNA of the subject as template;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14, wherein the primers amplify DNA of the microsatellite locus 11401m4 using genomic DNA of the subject as template;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16, wherein the primers amplify DNA of the microsatellite locus 441m6 using genomic DNA of the subject as template;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18, wherein the primers amplify DNA of the microsatellite locus 11292m4 using genomic DNA of the subject as template;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20, wherein the primers amplify DNA of the microsatellite locus 8499m4 using genomic DNA of the subject as template;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22, wherein the primers amplify DNA of the microsatellite locus 7203m5 using genomic DNA of the subject as template;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26, wherein the primers amplify DNA of the microsatellite locus 12130m5 using genomic DNA of the subject as template;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28, wherein the primers amplify DNA of the microsatellite locus 4546m2 using genomic DNA of the subject as template;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30, wherein the primers amplify DNA of the microsatellite locus 9079m3 using genomic DNA of the subject as template;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32, wherein the primers amplify DNA of the microsatellite locus 11192m4 using genomic DNA of the subject as template;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34, wherein the primers amplify DNA of the microsatellite locus 8010m6 using genomic DNA of the subject as template;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36, wherein the primers amplify DNA of the microsatellite locus 12198m3 using genomic DNA of the subject as template; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38, wherein the primers amplify DNA of the microsatellite locus 7850m4 using genomic DNA of the subject as template.

In the primer pair for amplifying a microsatellite locus in the genome of Acropora genus, the primer pair may be selected from the group consisting of:

(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer 5 comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer 15 comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer 25 comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer 35 comprising a nucleotide sequence of SEQ ID NO:36; and
(xix) a primer comprising a nucleotide sequence of SEQ ID NO:37 and a primer comprising a nucleotide sequence of SEQ ID NO:38.

In the primer pair for amplifying a microsatellite locus in the genome of Acropora genus, one of the primer pair may further comprise a nucleotide sequence of SEQ ID NO:39 (M13 primer) at its 5′ end.

In one aspect of the present invention, there is provided a method for identifying a subject of Acropora genus to an Acropora species, comprising of: detecting one or more microsatellite loci in the genome of the subject, wherein said microsatellite loci are listed in Table 1 or are selected from the group consisting of:

(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26;
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28;
(xv) a microsatellite locus 9079m3 having a 5′ flanking region that comprises a nucleotide sequence of SEQ ID NO:29 and a 3′ flanking region that comprises a nucleotide sequence complementary to SEQ ID NO:30;
(xvi) a microsatellite locus 11192m4 having a 5′ flanking region that comprises a nucleotide sequence of SEQ ID NO:31 and a 3′ flanking region that comprises a nucleotide sequence complementary to SEQ ID NO:32;
(xvii) a microsatellite locus 8010m6 having a 5′ flanking region that comprises a nucleotide sequence of SEQ ID NO:33 and a 3′ flanking region that comprises a nucleotide sequence complementary to SEQ ID NO:34;
(xviii) a microsatellite locus 12198m3 having a 5′ flanking region that comprises a nucleotide sequence of SEQ ID NO:35 and a 3′ flanking region that comprises a nucleotide sequence complementary to SEQ ID NO:36; and
(xix) a microsatellite locus 7850m4 having a 5′ flanking region that comprises a nucleotide sequence of SEQ ID NO:37 and a 3′ flanking region that comprises a nucleotide sequence complementary to SEQ ID NO:38.

In the method for identifying a subject of Acropora genus to an Acropora species, the detection of a microsatellite locus in the genome of the subject may be performed by using one or more primer pairs selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26;
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28;
(xv) a primer comprising at least 17 consecutive nucleotides having at least 90% sequence identity to a nucleotide sequence of SEQ ID NO:29 and a primer comprising at least 17 consecutive nucleotides having at least 90% sequence identity to a nucleotide sequence complementary to SEQ ID NO:30;
(xvi) a primer comprising at least 17 consecutive nucleotides having at least 90% sequence identity to a nucleotide sequence of SEQ ID NO:31 and a primer comprising at least 17 consecutive nucleotides having at least 90% sequence identity to a nucleotide sequence complementary to SEQ ID NO:32;
(xvii) a primer comprising at least 17 consecutive nucleotides having at least 90% sequence identity to a nucleotide sequence of SEQ ID NO:33 and a primer comprising at least 17 consecutive nucleotides having at least 90% sequence identity to a nucleotide sequence complementary to SEQ ID NO:34;
(xviii) a primer comprising at least 17 consecutive nucleotides having at least 90% sequence identity to a nucleotide sequence of SEQ ID NO:35 and a primer comprising at least 17 consecutive nucleotides having at least 90% sequence identity to a nucleotide sequence complementary to SEQ ID NO:36; and
(xix) a primer comprising at least 17 consecutive nucleotides having at least 90% sequence identity to a nucleotide sequence of SEQ ID NO:37 and a primer comprising at least 17 consecutive nucleotides having at least 90% sequence identity to a nucleotide sequence complementary to SEQ ID NO:38.

In the method for identifying a subject of Acropora genus to an Acropora species, the detection of a microsatellite locus in the genome of the subject may be performed by using one or more primer pairs selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2, wherein the primers amplify DNA of the microsatellite locus 8346m3 using genomic DNA of the subject as template;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4, wherein the primers amplify DNA of the microsatellite locus 7961m4 using genomic DNA of the subject as template;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6, wherein the primers amplify DNA of the microsatellite locus 11745m3 using genomic DNA of the subject as template;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8, wherein the primers amplify DNA of the microsatellite locus 12406m3 using genomic DNA of the subject as template;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10, wherein the primers amplify DNA of the microsatellite locus 11543m5 using genomic DNA of the subject as template;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12, wherein the primers amplify DNA of the microsatellite locus 530m4 using genomic DNA of the subject as template;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14, wherein the primers amplify DNA of the microsatellite locus 11401m4 using genomic DNA of the subject as template;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16, wherein the primers amplify DNA of the microsatellite locus 441m6 using genomic DNA of the subject as template;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18, wherein the primers amplify DNA of the microsatellite locus 11292m4 using genomic DNA of the subject as template;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20, wherein the primers amplify DNA of the microsatellite locus 8499m4 using genomic DNA of the subject as template;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22, wherein the primers amplify DNA of the microsatellite locus 7203m5 using genomic DNA of the subject as template;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26, wherein the primers amplify DNA of the microsatellite locus 12130m5 using genomic DNA of the subject as template;
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28, wherein the primers amplify DNA of the microsatellite locus 4546m2 using genomic DNA of the subject as template;
(xv) a primer comprising at least 17 consecutive nucleotides having at least 90% sequence identity to a nucleotide sequence of SEQ ID NO:29 and a primer comprising at least 17 consecutive nucleotides having at least 90% sequence identity to a nucleotide sequence complementary to SEQ ID NO:30, wherein the primers amplify DNA of the microsatellite locus 9079m3 using genomic DNA of the subject as template;
(xvi) a primer comprising at least 17 consecutive nucleotides having at least 90% sequence identity to a nucleotide sequence of SEQ ID NO:31 and a primer comprising at least 17 consecutive nucleotides having at least 90% sequence identity to a nucleotide sequence complementary to SEQ ID NO:32, wherein the primers amplify DNA of the microsatellite locus 11192m4 using genomic DNA of the subject as template;
(xvii) a primer comprising at least 17 consecutive nucleotides having at least 90% sequence identity to a nucleotide sequence of SEQ ID NO:33 and a primer comprising at least 17 consecutive nucleotides having at least 90% sequence identity to a nucleotide sequence complementary to SEQ ID NO:34, wherein the primers amplify DNA of the microsatellite locus 8010m6 using genomic DNA of the subject as template;
(xviii) a primer comprising at least 17 consecutive nucleotides having at least 90% sequence identity to a nucleotide sequence of SEQ ID NO:35 and a primer comprising at least 17 consecutive nucleotides having at least 90% sequence identity to a nucleotide sequence complementary to SEQ ID NO:36, wherein the primers amplify DNA of the microsatellite locus 12198m3 using genomic DNA of the subject as template; and
(xix) a primer comprising at least 17 consecutive nucleotides having at least 90% sequence identity to a nucleotide sequence of SEQ ID NO:37 and a primer comprising at least 17 consecutive nucleotides having at least 90% sequence identity to a nucleotide sequence complementary to SEQ ID NO:38, wherein the primers amplify DNA of the microsatellite locus 7850m4 using genomic DNA of the subject as template.

In the method for identifying a subject of Acropora genus to an Acropora species, the one or more primer pairs may be selected from the group consisting of:

(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer comprising a nucleotide sequence of SEQ ID NO:36; and
(xix) a primer comprising a nucleotide sequence of SEQ ID NO:37 and a primer comprising a nucleotide sequence of SEQ ID NO:38.

In the method for identifying a subject of Acropora genus to an Acropora species, one of the primer pair may further comprise a nucleotide sequence of SEQ ID NO: 39 (M13 primer) at its 5′ end.

In another aspect of the present invention, there is provided a primer pair for amplifying a microsatellite locus in the genome of an Acropora species, wherein said microsatellite locus is selected from Table 1 or is selected from the group consisting of:

(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26;
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28;
(xv) a microsatellite locus 9079m3 having a 5′ flanking region that comprises a nucleotide sequence of SEQ ID NO:29 and a 3′ flanking region that comprises a nucleotide sequence complementary to SEQ ID NO:30;
(xvi) a microsatellite locus 11192m4 having a 5′ flanking region that comprises a nucleotide sequence of SEQ ID NO:31 and a 3′ flanking region that comprises a nucleotide sequence complementary to SEQ ID NO:32;
(xvii) a microsatellite locus 8010m6 having a 5′ flanking region that comprises a nucleotide sequence of SEQ ID NO:33 and a 3′ flanking region that comprises a nucleotide sequence complementary to SEQ ID NO:34;
(xviii) a microsatellite locus 12198m3 having a 5′ flanking region that comprises a nucleotide sequence of SEQ ID NO:35 and a 3′ flanking region that comprises a nucleotide sequence complementary to SEQ ID NO:36; and
(xix) a microsatellite locus 7850m4 having a 5′ flanking region that comprises a nucleotide sequence of SEQ ID NO:37 and a 3′ flanking region that comprises a nucleotide sequence complementary to SEQ ID NO:38.

In the primer pair for amplifying a microsatellite locus in the genome of an Acropora species, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26;
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28;
(xv) a primer comprising at least 17 consecutive nucleotides having at least 90% sequence identity to a nucleotide sequence of SEQ ID NO:29 and a primer comprising at least 17 consecutive nucleotides having at least 90% sequence identity to a nucleotide sequence complementary to SEQ ID NO:30;
(xvi) a primer comprising at least 17 consecutive nucleotides having at least 90% sequence identity to a nucleotide sequence of SEQ ID NO:31 and a primer comprising at least 17 consecutive nucleotides having at least 90% sequence identity to a nucleotide sequence complementary to SEQ ID NO:32;
(xvii) a primer comprising at least 17 consecutive nucleotides having at least 90% sequence identity to a nucleotide sequence of SEQ ID NO:33 and a primer comprising at least 17 consecutive nucleotides having at least 90% sequence identity to a nucleotide sequence complementary to SEQ ID NO:34;
(xviii) a primer comprising at least 17 consecutive nucleotides having at least 90% sequence identity to a nucleotide sequence of SEQ ID NO:35 and a primer comprising at least 17 consecutive nucleotides having at least 90% sequence identity to a nucleotide sequence complementary to SEQ ID NO:36; and
(xix) a primer comprising at least 17 consecutive nucleotides having at least 90% sequence identity to a nucleotide sequence of SEQ ID NO:37 and a primer comprising at least 17 consecutive nucleotides having at least 90% sequence identity to a nucleotide sequence complementary to SEQ ID NO:38.

In the primer pair for amplifying a microsatellite locus in the genome of an Acropora species, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2, wherein the primers amplify DNA of the microsatellite locus 8346m3 using genomic DNA of the subject as template;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4, wherein the primers amplify DNA of the microsatellite locus 7961m4 using genomic DNA of the subject as template;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6, wherein the primers amplify DNA of the microsatellite locus 11745m3 using genomic DNA of the subject as template;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8, wherein the primers amplify DNA of the microsatellite locus 12406m3 using genomic DNA of the subject as template;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10, wherein the primers amplify DNA of the microsatellite locus 11543m5 using genomic DNA of the subject as template;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12, wherein the primers amplify DNA of the microsatellite locus 530m4 using genomic DNA of the subject as template;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14, wherein the primers amplify DNA of the microsatellite locus 11401m4 using genomic DNA of the subject as template;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16, wherein the primers amplify DNA of the microsatellite locus 441m6 using genomic DNA of the subject as template;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18, wherein the primers amplify DNA of the microsatellite locus 11292m4 using genomic DNA of the subject as template;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20, wherein the primers amplify DNA of the microsatellite locus 8499m4 using genomic DNA of the subject as template;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22, wherein the primers amplify DNA of the microsatellite locus 7203m5 using genomic DNA of the subject as template;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26, wherein the primers amplify DNA of the microsatellite locus 12130m5 using genomic DNA of the subject as template;
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28, wherein the primers amplify DNA of the microsatellite locus 4546m2 using genomic DNA of the subject as template;
(xv) a primer comprising at least 17 consecutive nucleotides having at least 90% sequence identity to a nucleotide sequence of SEQ ID NO:29 and a primer comprising at least 17 consecutive nucleotides having at least 90% sequence identity to a nucleotide sequence complementary to SEQ ID NO:30, wherein the primers amplify DNA of the microsatellite locus 9079m3 using genomic DNA of the subject as template;
(xvi) a primer comprising at least 17 consecutive nucleotides having at least 90% sequence identity to a nucleotide sequence of SEQ ID NO:31 and a primer comprising at least 17 consecutive nucleotides having at least 90% sequence identity to a nucleotide sequence complementary to SEQ ID NO:32, wherein the primers amplify DNA of the microsatellite locus 11192m4 using genomic DNA of the subject as template;
(xvii) a primer comprising at least 17 consecutive nucleotides having at least 90% sequence identity to a nucleotide sequence of SEQ ID NO:33 and a primer comprising at least 17 consecutive nucleotides having at least 90% sequence identity to a nucleotide sequence complementary to SEQ ID NO:34, wherein the primers amplify DNA of the microsatellite locus 8010m6 using genomic DNA of the subject as template;
(xviii) a primer comprising at least 17 consecutive nucleotides having at least 90% sequence identity to a nucleotide sequence of SEQ ID NO:35 and a primer comprising at least 17 consecutive nucleotides having at least 90% sequence identity to a nucleotide sequence complementary to SEQ ID NO:36, wherein the primers amplify DNA of the microsatellite locus 12198m3 using genomic DNA of the subject as template; and
(xix) a primer comprising at least 17 consecutive nucleotides having at least 90% sequence identity to a nucleotide sequence of SEQ ID NO:37 and a primer comprising at least 17 consecutive nucleotides having at least 90% sequence identity to a nucleotide sequence complementary to SEQ ID NO:38, wherein the primers amplify DNA of the microsatellite locus 7850m4 using genomic DNA of the subject as template.

In the primer pair for amplifying a microsatellite locus in the genome of an Acropora species, the primer pair may be selected from the group consisting of:

(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer comprising a nucleotide sequence of SEQ ID NO:36; and
(xix) a primer comprising a nucleotide sequence of SEQ ID NO:37 and a primer comprising a nucleotide sequence of SEQ ID NO:38.

In the primer pair for amplifying a microsatellite locus in the genome of Acropora species, one of the primer pair may further comprise a nucleotide sequence of SEQ ID NO:39 (M13 primer) at its 5′ end.

In another aspect of the present invention, there is provided a method for identifying an allele of a microsatellite locus which exhibits PCR size polymorphism in different genotypes of an Acropora species, comprising of:

identifying a microsatellite locus whose PCR fragment size, amplified with genomic DNA derived from a first coral colony of an Acropora species as template, is different from the PCR fragment size amplified with genomic DNA derived from a second coral colony of the Acropora species, wherein said microsatellite locus is selected from the group consisting of:

(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28.

In the kit for determining a PCR size polymorphic allele of a microsatellite locus of an Acropora species, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28.

In the kit for determining a PCR size polymorphic allele of a microsatellite locus of an Acropora species, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2, wherein the primers amplify DNA of the microsatellite locus 8346m3 using genomic DNA of the subject as template;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4, wherein the primers amplify DNA of the microsatellite locus 7961m4 using genomic DNA of the subject as template;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6, wherein the primers amplify DNA of the microsatellite locus 11745m3 using genomic DNA of the subject as template;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8, wherein the primers amplify DNA of the microsatellite locus 12406m3 using genomic DNA of the subject as template;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10, wherein the primers amplify DNA of the microsatellite locus 11543m5 using genomic DNA of the subject as template;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12, wherein the primers amplify DNA of the microsatellite locus 530m4 using genomic DNA of the subject as template;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14, wherein the primers amplify DNA of the microsatellite locus 11401m4 using genomic DNA of the subject as template;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16, wherein the primers amplify DNA of the microsatellite locus 441m6 using genomic DNA of the subject as template;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18, wherein the primers amplify DNA of the microsatellite locus 11292m4 using genomic DNA of the subject as template;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20, wherein the primers amplify DNA of the microsatellite locus 8499m4 using genomic DNA of the subject as template;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22, wherein the primers amplify DNA of the microsatellite locus 7203m5 using genomic DNA of the subject as template;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26, wherein the primers amplify DNA of the microsatellite locus 12130m5 using genomic DNA of the subject as template; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28, wherein the primers amplify DNA of the microsatellite locus 4546m2 using genomic DNA of the subject as template.

In the kit for determining a PCR size polymorphic allele of a microsatellite locus of an Acropora species, the primer pair may be selected from the group consisting of:

(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26; and
(xiv) a primer comprising a nucleotide sequence of SEQ ID NO:27 and a primer comprising a nucleotide sequence of SEQ ID NO:28.

In one more aspect of the present invention, there is provided a method for quantifying an index of level of chimerism of a chimeric coral colony restored by transplanting a first coral colony of an Acropora species or a part thereof with a second coral colony in need of restoration that belongs to the same species as the first coral colony, comprising of:

(a) identifying one or more microsatellite loci whose PCR fragment size, amplified with genomic DNA derived from the first coral colony as template, is different from the PCR fragment size amplified with genomic DNA derived from the second coral colony, wherein said microsatellite loci are selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28;
(b) determining the molar percentages of the genomic DNA for the allele representing the first and second coral colonies, and
(c) providing the molar percentage of the PCR fragment representing the first coral colony as the index of level of chimerism of the chimeric coral colony restored.

In one more aspect of the present invention, there is provided a kit for quantifying an index of level of chimerism of a chimeric coral colony restored by transplanting a first coral colony of an Acropora species or a part thereof with a second coral colony in need of restoration that belongs to the same species as the first coral colony, comprising

a primer pair which amplify a microsatellite locus in the genome of the subject, wherein said microsatellite loci are selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28.

In the kit for quantifying an index of a chimeric coral colony restored by transplanting a first coral colony of an Acropora species into a second coral colony in need of restoration that belongs to the same species as the first coral colony, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28.

In the kit for quantifying an index of level of chimerism of the chimeric coral colony restored by transplanting the first coral colony of an Acropora species or a part thereof with the second coral colony in need of restoration that belongs to the same species as the first coral colony, the primer pair may be selected from the group consisting of:

(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2, wherein the primers amplify DNA of the microsatellite locus 8346m3 using genomic DNA of the subject as template;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4, wherein the primers amplify DNA of the microsatellite locus 7961m4 using genomic DNA of the subject as template;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6, wherein the primers amplify DNA of the microsatellite locus 11745m3 using genomic DNA of the subject as template;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8, wherein the primers amplify DNA of the microsatellite locus 12406m3 using genomic DNA of the subject as template;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10, wherein the primers amplify DNA of the microsatellite locus 11543m5 using genomic DNA of the subject as template;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12, wherein the primers amplify DNA of the microsatellite locus 530m4 using genomic DNA of the subject as template;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14, wherein the primers amplify DNA of the microsatellite locus 11401m4 using genomic DNA of the subject as template;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16, wherein the primers amplify DNA of the microsatellite locus 441m6 using genomic DNA of the subject as template;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18, wherein the primers amplify DNA of the microsatellite locus 11292m4 using genomic DNA of the subject as template;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20, wherein the primers amplify DNA of the microsatellite locus 8499m4 using genomic DNA of the subject as template;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22, wherein the primers amplify DNA of the microsatellite locus 7203m5 using genomic DNA of the subject as template;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24, wherein the primers amplify DNA of the microsatellite locus 10366m5 using genomic DNA of the subject as template;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26, wherein the primers amplify DNA of the microsatellite locus 12130m5 using genomic DNA of the subject as template; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28, wherein the primers amplify DNA of the microsatellite locus 4546m2 using genomic DNA of the subject as template.

In the kit for quantifying the index of level of chimerism of the chimeric coral colony restored by transplanting the first coral colony of an Acropora species or a part thereof with the second coral colony in need of restoration that belongs to the same species as the first coral colony, the primer pair may be selected from the group consisting of:

(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26; and
(xiv) a primer comprising a nucleotide sequence of SEQ ID NO:27 and a primer comprising a nucleotide sequence of SEQ ID NO:28.

### BRIEF DESCRIPTION OF THE DRAWINGS

FIG. 1. Phylogenetic relationship of Acropora corals inferred from molecular markers. Acropora species used in this study (A. tenuis, A. digitifera and A. hyacinthus) are shown. Modified from Van Oppen et al. (2001) and Marquez et al. (2002).

### DETAILED DESCRIPTION OF THE INVENTION

Unless otherwise indicated, all numbers such as those expressing weight percents of ingredients, dimensions, and values for certain physical properties used in the specification and claims are to be understood as being modified in all instances by the term “about.” It should also be understood that the specific numerical values used in the specification and claims form additional embodiments of the invention. Efforts have been made to ensure the accuracy of the numerical values disclosed in the Examples. Any measured numerical value, however, can inherently contain certain errors resulting from the standard deviation found in its respective measuring technique.

As used herein the use of the indefinite article “a” or “an” means “at least one,” and should not be limited to “only one” unless explicitly indicated to the contrary. Thus, for example, reference to “a metal catalyst” includes embodiments having one, two or more metal catalysts, unless the context clearly indicates otherwise.

The phrase “identifying a subject to Acropora genus” or “identifying a subject of Acropora genus to an Acropora species,” as used herein, means that a subject of an Acropora genus is determined as classified to a particular species of Acropora or the phrase means for distinguishing a particular Acropora species to which a subject belongs. For each of the particular species of Acropora, a reference pattern of PCR fragment size polymorphism for the one or more of microsatellite loci of the present invention is determined. PCR amplification using, as template, genomic DNA of a coral sample to be identified is carried out for each of the microsatellite loci to obtain a sample pattern of PCR fragment size polymorphism. When the sample pattern matches with one of the reference pattern, then the coral sample is identified to belong to the same species as the species of the referenced pattern.

The term “subject” as used herein is meant for a biological material which includes, but not limited to, such multicellular coral organism as a larva, a planula, a polyp and a colony of a coral, an individual coral cell comprising the multicellular coral organism such as a neuron, a nematocyst and an epidermal cell, a tissue or an organ comprising of the individual coral cells such as tentacle, an epidermis, a mesoglea and a calicle, and a unicellular organism such as a gamete, a sperm and a gygote,

The term “PCR” as used herein is meant that the polymerase chain reaction technology is applied for identification of a coral species in Acropora genus. Preparation of genomic DNA from biological material of Acropora coral origin any commercially available kit including, but not limited to, a DNeasy kit of QIAGEN N.V. of The Netherlands. Experimental techniques are described in laboratory manuals including, but not limited to, “Molecular Cloning: A Laboratory Manual,” (Fourth Edition, by M. R. Green and J. Sambrook, Cold Spring Harbor Laboratory Press, 2012).

The phrase “comprising at least 17 consecutive nucleotides,” as used herein, means that the primer of the present invention has 17 bases or more, for example, 17, 18 19, 20, 21, 22, 23, 24, 25, 26, 27, 28, 29, 30 or more nucleotides of the particular nucleotide sequence set forth in a referred sequence identifier, without any intervening nucleotide.

The phrase “having at least 90% sequence identity to a sequence,” as used herein, means that the primer of the present invention has 90% or more, for example, 93, 95, 97, 99% or more, or 100% sequence identity to a particular nucleotide sequence associated with a particular sequence identifier. The sequence identity is determined by aligning the two sequences to be compared as described below, determining the number of identical bases in the aligned portion, dividing that number by the total number of bases in the inventive (queried) sequence, and multiplying the result by 100. Polynucleotide and polypeptide sequences may be aligned, and the percentage of identical residues in a specified region may be determined against another polynucleotide or polypeptide, using a publicly available computer algorithm. Two exemplary algorithms for aligning and identifying the similarity of polynucleotide sequences are the BLASTN and FASTA algorithms. The computer algorithms BLASTN and FASTA are available on the Internet such as The National Center for Biotechnology Information (NCBI) of the U.S. The use of the BLASTN algorithm is described in the publication of Altschul, et al., Nucleic Acids Res. 25: 3389-3402, 1997. The use of the FASTA algorithm is described in Pearson and Lipman, Proc. Natl. Acad. Sci. USA 85:2444-2448, 1988; and Pearson, Methods in Enzymol. 183: 63-98, 1990.

The phrase “having at least 90% sequence identity to a sequence,” as used herein, may be replaced with a phrase “having a nucleotide sequence wherein one or a few nucleotides are deleted, substituted, or added to a particular nucleotide sequence associated with a particular sequence identifier.” The wording “a few nucleotides” means for two, three four, five, six, seven, eight, nine or ten nucleotides. A pair of primers defined with the phrase “having a nucleotide sequence wherein one or a few nucleotides are deleted, substituted, or added to a particular nucleotide sequence associated with a particular sequence identifier” may also be defined so that the primers amplify DNA of a microsatellite locus disclosed in the present application.

The phrase “identifying an allele of a microsatellite locus which exhibits PCR size polymorphism in different genotypes of an Acropora species,” as used herein, means that the size of PCR fragment amplified with genomic DNA of cells derived from a gamete of an Acropora species is distinct from the size of PCR fragment amplified with genomic DNA of cells derived from a different gamete of the Acropora. The particular microsatellite locus which exhibits PCR fragment size polymorphism in a particular combination of coral colonies of interest may be called as an “informative” locus. Thus, the informative locus may exhibit PCR size polymorphism between one colony of an Acropora species and another colony of the Acropora species may be employed to determine whether the colony is composed of cells derived from a single gamete or the colony is chimeric, that is, composed of cells derived from a plurality of gametes.

The phrase “determining a PCR size polymorphic allele of a microsatellite locus of an Acropora species,” as used herein, means that a coral sample is analysed by PCR amplification to determine whether the coral sample has a particular allele of the microsatellite locus that has a specific PCR fragment size which is distinct from other allele of the same microsatellite locus. A method and kit for determining a PCR size polymorphic allele of a microsatellite locus of an Acropora species comprises a primer pair that is selected by the method of present invention for identifying an allele of a microsatellite locus which exhibits PCR size polymorphism in different genotypes of an Acropora species. The method and kit for determining a PCR size polymorphic allele of a microsatellite locus of an Acropora species is useful in determining whether an entire colony of an Acropora species is composed of cells derived from a single gamete or the colony is chimeric. The method and kit is also useful in quantifying an index of chimeric status of a coral colony. The method and kit is also useful in analyzing genetic composition of coral colony or coral population in a wild or artificial habitat.

The phrase “an index of chimeric level” means for a quantitative parameter of relative contribution of descendent cells of a first coral colony, or a donor colony, in the entire transplanted colony that is comprised of the descendents of the first coral colony and a second coral colony, or a recipient colony, which had been in need of restoration. Alternatively, the index of chimeric level means for relative percentage of cells derived from the first colony in the total cells of the transplanted colony which is comprised of cells derived from the second colony and which may also be comprised of successfully engrafted cells derived from the first colony. The index of chimeric level is determined quantitatively by identifying an informative microsatellite locus for a particular combination of donor and recipient colonies of the same coral species of Acropora genus, determining the molar percentage of the genomic DNA for the alleles representing the first and second coral colonies, and providing the molar percentage of the PCR fragment representing the first coral colony as the index of level of chimerism of the chimeric coral colony restored. The amplification of the genomic DNA for the alleles representing the first and second coral colonies may be carried out with any PCR technique well-known to those skilled in the art, such as Wang L.-J. (2002), with a reaction condition by which the molar percentage of the genomic DNA for the alleles is reproducibly determined with reference to a calibration curve, or a mixing DNA curve. Based on the size of the PCR fragments amplified from the particular alleles of the informative microsatellite locus, the PCR fragments amplified from each allele may be separated with any well-known technique for size separation such as conventional gel electrophoresis, or a capillary electrophoresis with, but not limited to, agarose or polyacrylamide gel as separation media. The amounts of the PCR fragments of each allele may be measured with fluorescence or laser-induced fluorescence based detection system in conjunction with fluor labeling systems. The calibration curve is produced by plotting relative amounts of the PCR fragments amplified with a mixture of genomic DNAs of the first and second colonies at different ratios. Typically, the ratios of the first and second colonies are set as any combination of ratios selected from the group consisting of 0:100, 5:95, 10:90, 15:85, 20:80, . . . , 40:60, 45:55, 50:50, 55:45, . . . , 80:20, 85:15, 90:10, 95:5, and 100:0. The molar percentage of the genomic DNA for the alleles are determined from the relative amounts of the PCR fragments amplified representing the first and second colonies with reference to the calibration curve. The molar percentage of the genomic DNA for the allele representing the first colony is calculated as 100× (relative molar content of PCR fragment representing the first colony)/(sum of relative molar contents of PCR fragments representing the first and second colonies). The molar percentages of the genomic DNA for the allele representing the second colony is calculated as 100× (relative molar content of PCR fragment representing the second colony)/(sum of relative molar contents of PCR fragments representing the first and second colonies).

The present invention is further illustrated by the following non-limiting examples.

Materials and Methods

Genomic DNA was isolated from an A. tenuis colony collected at Sesoko Island, Okinawa, Japan, under Okinawa prefectural permit (Number: 24-48), using the guanidinium reagent, CHAOS (Fukami et al., 2000). We sequenced 250 bp paired end reads using a MiSeq sequencer (Illumina) according to manufacturer's instructions. Low quality bases (Phred quality value, QV≧20) were trimmed from raw data and read pairs of at least 80 bp were retained using SolexaQA (Cox et al., 2010). We used PAL_FINDER (Castoe et al., 2012) for detection of simple sequence repeats (SSRs) and PCR primer design from paired end sequencing data. In order to select microsatellite loci that may be highly variable, we selected primer pairs amplifying longer repeat stretches (thresholds: 2 mer; 15 repeats more, 3 mer; 10, 4 mer; 7, 5 mer; 5 and 6 mer; 4, respectively). To remove primers originating from DNA of the symbiotic Symbiodinium, nucleotide sequences of both primers in each pair were mapped to the recently decoded A. digitifera genome (Shinzato et al., 2011), using Symbiodinium-free sperm DNA, and employing BLASTN software (Altschul et al., 1990). In addition, primer pairs from which at least one primer was mapped uniquely to the A. digitifera genome were selected in order to avoid selecting primer pairs that could produce nonspecific PCR amplification.

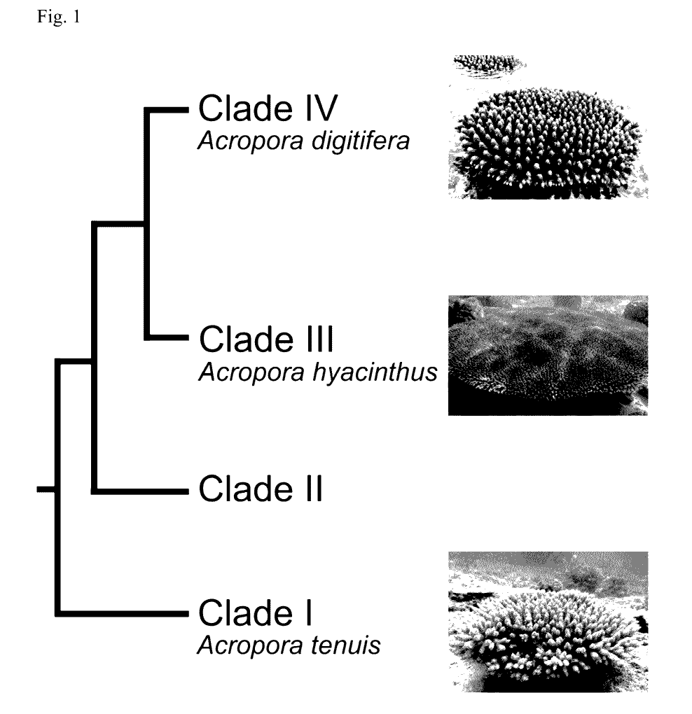

For fragment analyses, 30 colonies of A. tenuis were collected at Sesoko Island, Okinawa, Japan, and 45 colonies of A. digitifera were collected in the Kerama Islands, Okinawa, Japan, respectively (Okinawa prefecture permit number: 24-48). To avoid multiple collections of colonies that could have been produced through asexual fragmentation or propagation, only colonies that were physically distinct and at least 2 m from other colonies were sampled. Genomic DNA was extracted using a DNeasy kit (QIAGEN). The reaction mixture (10 μL) contained template DNA (<1 ng/L), AmpliTaq Gold 360 Master Mix (Qiagen), and three primers for each locus: a non-tailed reverse primer (0.1 μM), a forward primer with an M13 Reverse (5′-CAGGAAACAGCTATGAC-3′) sequence tail (0.5 μM), and an M13 Reverse primer (0.5 μM) fluorescently labeled with FAM, based on the method of Schuelke (2000). PCR cycling conditions were 15 min at 95° C., followed by 32 cycles of 30 s at 94° C., 90 s at 58° C. (all loci), and 60 s at 72° C., with an extension of 30 min at 60° C. in the final cycle. In addition to A. tenuis and A. digitifera, we also used A. hyacinthus (Clade III) (FIG. 1) (Marquez et al., 2002) genomic DNA to confirm PCR amplification (data not shown). PCR products from A. tenuis and A. digitifera were identified and analyzed with the ABI 3130 capillary sequencer (Applied Biosystems) and GeneMapper v4.1 (Applied Biosystems). The number of alleles and observed and expected heterozygosities were calculated and the probability of deviation from Hardy-Weinberg equilibrium (HWE) was tested for each locus and species, using GenAlEx ver. 6.5 (Peakall and Smouse, 2012). Linkage disequilibrium between the loci was tested after Bonferroni correction (P<0.05) using Genepop v4.2 at http://genepop.curtin.edu.au/index.html (Raymond and Rousset, 1995; Rousset, 2008).

Results and Discussion

We obtained 6,327,391,737 bp (12,802,836 read pairs) of raw sequence data from A. tenuis genomic DNA. From those we selected high quality 2,534,049,158 bp (6,783,510 read pairs), which were used for microsatellite detection and primer design. Primer pairs (7,200) were produced by PAL_FINDER. In order to eliminate primer pairs that could have produced non-specific PCR amplification and that originated from symbiotic Symbiodinium, we selected pairs from which at least one primer sequence was unique to the A. digitifera genome sequence. Subsequently 141 primer pairs were selected. Among those, we confirmed that 74 pairs could produce PCR amplicons in three Acropora species (A. tenuis, A. digitifera and A. hyacinthus) and performed fragment analyses. We identified fourteen polymorphic nuclear microsatellite DNA makers that did not show significant deviation from HWE after applying Bonferroni correction (P<0.05) in both A. tenuis and A. digitifera. Four markers (9079m3, 11192m4, 8010m6 and 12198m3) showed significant deviation from HWE in A. digitifera, but not in A. tenuis, and one marker (7805m4) showed significant deviation from HWE in A. tenuis, but not in A. digitifera (Table 1). We confirmed that no previously reported Acropora microsatellite primer sequences (Baums et al., 2005; Van Oppen et al., 2007; Nakajima et al., 2009; Concepcion et al., 2010) were detected in the PCR amplicon sequences, indicating that all microsatellite loci identified in this study are novel. The number of alleles per locus ranged from 3 to 14 in A. tenuis and 2 to 13 in A. digitifera, respectively (Table 1). Observed and expected heterozygosities ranged from 0.192 to 0.933 and 0.341 to 0.892 in A. tenuis and 0.200 to 0.911 and 0.241 to 0.862 in A. digitifera, respectively (Table 1). Although only the linkage disequilibrium between 11401m4 and 11745m3 was significant in A. digitifera, linkage disequilibrium between 11 loci combinations (11401m4-11745m3, 11401m4-11543m5, 11401m4-7203m5, 11401m4-12406m3, 11745m3-12406m3, 12406m3-10366m5, 441m6-12406m3, 530m4-11543m5, 530m4-8346m3, 7203m5-11745m3 and 8346m3-11543m5) was significant in A. tenuis. Alignment of the A. tenuis nucleotide sequences to the A. digitifera genome revealed high nucleotide conservation between A. tenuis and A. digitifera (about 93%, BLASTN, 1e-5, alignment length longer than 100 bp), indicating high genomic similarity between the two species. In addition, all microsatellite loci are located in different scaffold sequences in the A. digitifera genome (Table 1), suggesting that the loci are evenly distributed across these Acropora genomes.

Table 1 shows characteristics of the 19 developed polymorphic microsatellite loci from A. tenuis and A. digitifera: locus name, repeat motif, primers sequence, number of alleles, size range, observed (Ho) and expected (He) heterozygotes and GenBank accession number. Numbers of alleles and Ho and He were calculated using GenAlEx (ver. 6.5; Peakall and Smouse, 2012). Asterisks indicate significant deviation from Hardy-Weinberg equilibrium after Bonferroni correction (P<0.05) using Genepop v4.2 (Raymond and Rousset, 1995; Rousset, 2008). Accession numbers in A. digitifera are the scaffold nucleotide sequence in which each locus is located.

TABLE 1

| | Repeat motif | | |
|---|---|---|---|
| Locus | A. tenuis | A. digitifera | Primer sequences (5′-3′) |
| 8346m3 | (ATT)₁₂ | (ATT)₇ | M13R-CGACAAAGATTGGAGACCC |
| | | | TTTCAATGCAGTGTGATTCC |
| 7961m4 | (AAAG)₇ | (AAAG)₃ | M13R-AAGCATCACCAAAACGGC |
| | | | TTACATTTGCGTCTCGGC |
| 11745m3 | (AAT)₁₆GATAATGAT(AAT)₁₈ | (AAT)₄ | M13R-TTCTGTTCGCGTGTTCCC |
| | | | TGTTCTGCCACTGGAGGG |
| 12406m3 | (AAC)₆ | (AAC)₇AGC(AAC)₃ | M13R-GGTGAAGTTGTCTCCGTCC |
| | | | TTTTCAGGCATATCAGGAGC |
| 11543m5 | (AAAAG)₂ | (AAAAG)₄ | M13R-TTCTGACACAGCCATGAACC |
| | | | CCCCTTTCCAAAATTCACC |
| 530m4 | (AATG)₃ GATG(AATG)₇ | (AATG)₃ | M13R-GTTCACAGGAGTGTTATGCC |
| | | | TGTCATTTCCACGTTGTCC |
| 11401m4 | (ATTT)₈ | (ATTT)₅ | M13R-TGCAGACAGAACCGAGAACG |
| | | | TGGGCCACGATTGTTACG |
| 441m6 | (CTCCGT)₄ | (CTCGGT)₃ | M13R-GCCTTCCGGAACTATCGC |
| | | | TCCCAAGATGGTGTCACC |
| 11292m4 | (AATG)₄ | (AATG)₇ | M13R-TGCGAATGGAGCTCTGG |
| | | | TCATTTCGTCCATTCATGC |
| 8499m4 | (CGGT)₅ | (CGGT)₇ | M13R-AAACCGTGGGTTAAGGGC |
| | | | CGATGGAATTATTCGCGG |
| 7203m5 | (AAAAT)₄ | (AAAAT)₅ | M13R-ATTTCCTCACCATTCCCC |
| | | | TGAGGGAAAACAACACTCC |
| 10366m5 | (AAAAC)₅ | (AAAAC)₂ | M13R-CAACGACTGAAAGGCAGC |
| | | | GGCTTTCGACTTTTATGTCC |
| 12130m5 | (AAAAC)₆ | AAAACAAAAAA(AAAAC)₂ | M13R-TGAGGGTAAAGGCGGACC |
| | | | TTTTGCTTTATCCGGATCG |
| 4546m2 | (AT)₂ | (AT)₂AATT(AT)₂AC(AT)₃ | M13R-TGTGCAATGAAAATTTCCCC |
| | | | CAGTTCCCTTGTTCCTGGG |

Deviation from HWE in A. digitifera

| | | | |
|---|---|---|---|
| 9079m3 | (TAA)₁₁ | (TAA)₁₂ | M13R-TTTCGTGTTATAGCTCCCG |
| | | | CCTGGCTTTTAATCTGAGG |
| 11192m4 | (AAAC)₇ | (AAAC)₆ | M13R-TGAGGACCCTCCCTTCC |
| | | | AGGCTGCATCTGGTTTCC |
| 8010m6 | (AAAGGG)₄ | (AAAGGG)₂ | M13R-ACGGTGTGGTAAAGCACG |
| | | | CACTTGACACCACGCTGC |
| 12198m3 | (TAA)₁₄ | (TAA)₇ | M13R-CATCTCCAAGGAACTTTGC |
| | | | TTCACGTTGTGTTTTGGC |

Deviation from HWE in A. tenuis

| | | | |
|---|---|---|---|
| 7850m4 | (AATC)₉ | (AATC)₄ | M13R-ATGCCTGCAAGTGTTTGG |
| | | | GTTTCTTTAACGTCACGCGTTGTCC |

| | No. of alleles | | Size range of | | | | | |
|---|---|---|---|---|---|---|---|---|
| | | A. | alleles (bp) | | Ho/He | | Accession No. | |
| Locus | A. tenuis | digitifera | A. tenuis | A. digitifera | A. tenuis | A. digitifera | A. tenuis | A. digitifera |
| 8346m3 | 8 | 6 | 162-213 | 186-210 | 0.933/0.794 | 0.733/0.720 | AB915217 | DF093698.1 |
| 7961m4 | 3 | 4 | 192-208 | 174-190 | 0.192/0.341 | 0.556/0.481 | AB915218 | DF093718.1 |
| 11745m3 | 14 | 5 | 216-269 | 171-183 | 0.900/0.859 | 0.644/0.598 | AB819219 | DF093961.1 |
| 12406m3 | 5 | 9 | 163-175 | 164-168 | 0.733/0.776 | 0.600/0.570 | AB915220 | DF093708.1 |
| 11543m5 | 3 | 4 | 132-142 | 125-143 | 0.483/0.469 | 0.200/0.224 | AB915221 | DF094105.1 |
| 530m4 | 6 | 4 | 269-405 | 382-398 | 0.571/0.546 | 0.533/0.662 | AB915222 | DF093793.1 |
| 11401m4 | 7 | 6 | 382-418 | 364-400 | 0.833/0.729 | 0.267/0.345 | AB915223 | DF093776.1 |
| 441m6 | 5 | 6 | 288-312 | 268-298 | 0.733/0.697 | 0.666/0.674 | AB915224 | DF094515.1 |
| 11292m4 | 7 | 9 | 443-495 | 466-502 | 0.633/0.647 | 0.733/0.756 | AB915225 | DF093646.1 |
| 8499m4 | 5 | 6 | 340-356 | 341-361 | 0.733/0.527 | 0.778/0.669 | AB915226 | DF096297.1 |
| 7203m5 | 5 | 2 | 279-319 | 293-298 | 0.448/0.552 | 0.311/0.346 | AB915227 | DF093797.1 |
| 10366m5 | 3 | 3 | 222-232 | 216-226 | 0.833/0.569 | 0.578/0.447 | AB915228 | DF095728.1 |
| 12130m5 | 4 | 5 | 248-283 | 265-295 | 0.893/0.538 | 0.302/0.268 | AB915229 | DF095024.1 |
| 4546m2 | 3 | 4 | 250-282 | 231-255 | 0.367/0.471 | 0.068/0.067 | AB915230 | DF093922.1 |

Deviation from HWE in A. digitifera

| | | | | | | | | |
|---|---|---|---|---|---|---|---|---|
| 9079m3 | 7 | 13 | 269-287 | 219-303 | 0.767/0.779 | 0.911*/0.862 | AB915231 | DF093956.1 |
| 11192m4 | 4 | 5 | 127-139 | 104-120 | 0.633/0.660 | 0.203*/0.490 | AB915232 | DF094226.1 |
| 8010m6 | 4 | 4 | 204-228 | 189-213 | 0.500/0.442 | 0.200*/0.241 | AB913233 | DF093908.1 |
| 12198m3 | 13 | 6 | 304-346 | 294-339 | 0.767/0.892 | 0.222*/0.619 | AB915234 | DF093689.1 |

Deviation from HWE in A. tenuis

| | | | | | | | | |
|---|---|---|---|---|---|---|---|---|
| 7850m4 | 9 | 6 | 225-265 | 229-253 | 0.414*/0.829 | 0.733/0.637 | AB915235 | DF093738.1 |

Since genome structures of A. digitifera and A. tenuis should be similar, this may reflect the significant A. tenuis population decrease after a massive 1998 bleaching event around Sesoko island (Loya et al., 2001). Thus, the population used in this study was new and recently recruited, possibly resulting in more loci combinations with significant linkage disequilibrium. Although not tested on a large number of Acropora species, fourteen primer pairs were shown to have no significant deviation from HWE in two phylogenetically distant species. These can be used for a variety of Acropora species and may provide powerful tools for Acropora population genetic studies.

We have recently confirmed that we could detect PCR amplification of the all fourteen markers for, not only A. tenuis and A. digitifera but other 24 Acropora species (Table 2).

Table 2 shows summary of application of the 14 microsatellite markers for 26 Acropora species. Locus (marker) names, Acropora species that we used are shown. N represents the number of individuals for each species. A circle indicates that we confirmed PCR amplification using the marker. Gray indicate that differences of PCR fragment sizes between individuals were observed in the same species.

TABLE 2

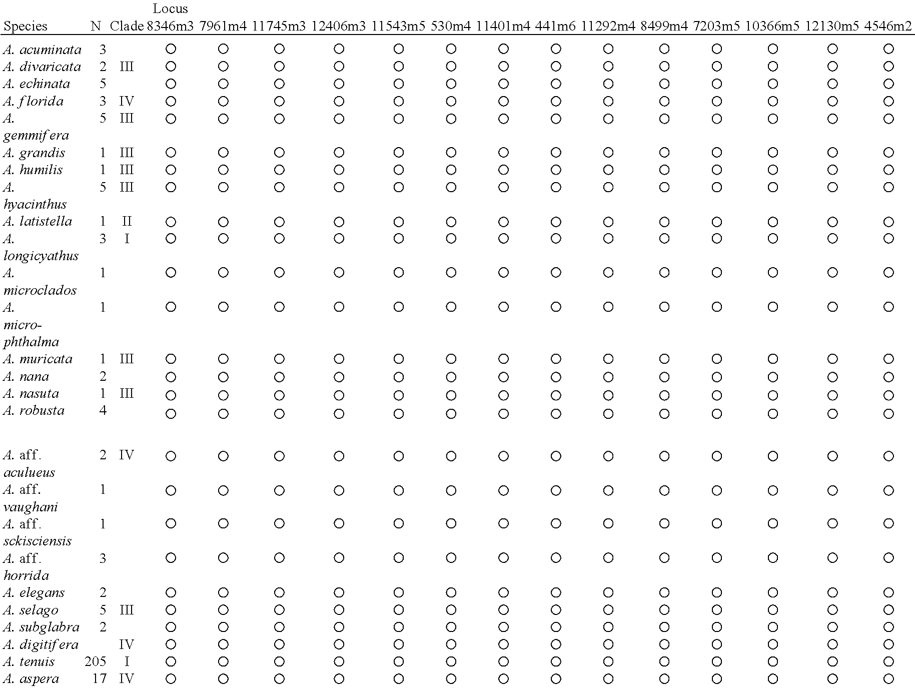

26 Species

We hope that they will contribute to establishment of reef conservation guidelines and coral reef transplantation and restoration.

It will be apparent to those skilled in the art that various modifications and alterations can be made to the present invention without departing from the scope and spirit of the invention. Thus, it is intended that the present invention cover the modifications and variations of this invention provided they come within the scope of the appended claims and their equivalents. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### REFERENCES

Altschul, S. F., Gish, W., Miller, W., Myers, E. W., and Lipman, D. J. (1990). Basic local alignment search tool. J Mol Biol 215, 403-410.
Alvarez-Filip, L., Carricart-Ganivet, J. P., Horta-Puga, G, and Iglesias-Prieto, R. (2013). Shifts in coral-assemblage composition do not ensure persistence of reef functionality. Sci Rep 3, 3486.
Baums, I. B., Hughes, C. R., and Hellberg, M. E. (2005). Mendelian microsatellite loci for the Caribbean coral Acropora palmata. Marine Ecology Progress Series 288, 115-127.
Castoe, T. A., Poole, A. W., De Koning, A. P., Jones, K. L., Tomback, D. F., Oyler-Mccance, S. J., Fike, J. A., Lance, S. L., Streicher, J. W., Smith, E. N., and Pollock, D. D. (2012). Rapid microsatellite identification from Illumina paired-end genomic sequencing in two birds and a snake. PLoS One 7, e30953.
Concepcion, G. T., Polato, N. R., Baums, I. B., and Toonen, R. J. (2010). Development of microsatellite markers from four Hawaiian corals: Acropora cytherea, Fungia scutaria, Montipora capitata and Porites lobata. Conservation Genetics Resources 2, 11-15.
Cox, M. P., Peterson, D. A., and Biggs, P. J. (2010). SolexaQA: At-a-glance quality assessment of Illumina second-generation sequencing data. BMC Bioinformatics 11, 485.
Fukami, H., Omori, M., and Hatta, M. (2000). Phylogenetic relationships in the coral family acroporidae, reassessed by inference from mitochondrial genes. Zoolog Sci 17, 689-696.
Hatta, M., Fukami, H., Wang, W., Omori, M., Shimoike, K., Hayashibara, T., Ina, Y, and Sugiyama, T. (1999). Reproductive and genetic evidence for a reticulate evolutionary history of mass-spawning corals. Mol Biol Evol 16, 1607-1613.
Hoegh-Guldberg, O., Mumby, P. J., Hooten, A. J., Steneck, R. S., Greenfield, P., Gomez, E., Harvell, C. D., Sale, P. F., Edwards, A. J., Caldeira, K., Knowlton, N., Eakin, C. M., Iglesias-Prieto, R., Muthiga, N., Bradbury, R. H., Dubi, A., and Hatziolos, M. E. (2007). Coral reefs under rapid climate change and ocean acidification. Science 318, 1737-1742.
Knowlton, N., Brainard, R. E., Fisher, R., Moews, M., Plaisance, L., and Caley, M. J. (2010). “Coral Reef Biodiversity,” in McIntyre A D (ed.). Life in the World's Oceans: Diversity, Distribution, and Abundance. Wiley-Blackwell, 65-79.
Loya, Y, Sakai, K., Yamazato, K., Nakano, Y, Sambali, H., and Van Woesik, R. (2001). Coral bleaching: the winners and the losers. Ecology Letters 4, 122-131.
Marquez, L. M., Van Oppen, M. J., Willis, B. L., Reyes, A., and Miller, D. J. (2002). The highly cross-fertile coral species, Acropora hyacinthus and Acropora cytherea, constitute statistically distinguishable lineages. Mol Ecol 11, 1339-1349.
Nakajima, Y, Nishikawa, A., Iguchi, A., and Sakai, K. (2009). Novel and cross-species amplifiable microsatellite markers in two Acropora species. Plankton Benthos Res 4, 38-41.
Peakall, R., and Smouse, P. E. (2012). GenAlEx 6.5: genetic analysis in Excel. Population genetic software for teaching and research—an update. Bioinformatics 28, 2537-2539.
Raymond, M., and Rousset, F. (1995). Genepop (Version-1.2)— Population-Genetics Software for Exact Tests and Ecumenicism. Journal of Heredity 86, 248-249.
Rousset, F. (2008). genepop′007: a complete re-implementation of the genepop software for Windows and Linux. Mol Ecol Resour 8, 103-106.
Schuelke, M. (2000). An economic method for the fluorescent labeling of PCR fragments. Nat Biotechnol 18, 233-234.
Shinzato, C., Shoguchi, E., Kawashima, T., Hamada, M., Hisata, K., Tanaka, M., Fujie, M., Fujiwara, M., Koyanagi, R., Ikuta, T., Fujiyama, A., Miller, D. J., and Satoh, N. (2011). Using the Acropora digitifera genome to understand coral responses to environmental change. Nature 476, 320-323.
Van Oppen, M. J., Mcdonald, B. J., Willis, B., and Miller, D. J. (2001). The evolutionary history of the coral genus Acropora (Scleractinia, Cnidaria) based on a mitochondrial and a nuclear marker: reticulation, incomplete lineage sorting, or morphological convergence? Mol Biol Evol 18, 1315-1329.
Van Oppen, M. J. H., Underwood, J. N., Muirhead, A. N., and Peplow, L. (2007). Ten microsatellite loci for the reef-building coral Acropora millepora (Cnidaria, Scleractinia) from the Great Barrier Reef, Australia. Molecular Ecology Notes 7, 436-438.
Veron, J. E. N. (1995). Corals in space and time: the biogeography and evolution of the Scleractinia. Ithaca: Comstock/Cornell.
Wallace, C. (1999). Staghorn corals of the world: a revision of the genus Acropora. Collingwood, Victoria, Australia: CSIRO PUBLISHING
Wang L.-J., Chou P., Gonzalez-Ryan L., Huang W., Haut P. R. Kletzel M. (2002). Evaluation of mixed hematopoietic chimerism in pediatric patients with leukemia after allogeneic stem cell transplantation by quantitative PCR analysis of variable number of tandem repeat and testis determination gene. Bone Marrow Transplant 2002; 29:51-6.

1. A method for identifying a subject to Acropora genus, comprising the steps of:
detecting one or more microsatellite loci in the genome of the subject, wherein said microsatellite loci are selected from the group consisting of:
(i) a microsatellite locus 8346m3 wherein its 5′ flanking region comprises a nucleotide sequence of SEQ ID NO:1 and its 3′ flanking region comprises a nucleotide sequence complementary to SEQ ID NO:2;
(ii) a microsatellite locus 7961m4 wherein its 5′ flanking region comprises a nucleotide sequence of SEQ ID NO:3 and its 3′ flanking region comprises a nucleotide sequence complementary to SEQ ID NO:4;
(iii) a microsatellite locus 11745m3 wherein its 5′ flanking region comprises a nucleotide sequence of SEQ ID NO:5 and its 3′ flanking region comprises a nucleotide sequence complementary to SEQ ID NO:6;
(iv) a microsatellite locus 12406m3 wherein its 5′ flanking region comprises a nucleotide sequence of SEQ ID NO:7 and its 3′ flanking region comprises a nucleotide sequence complementary to SEQ ID NO:8;
(v) a microsatellite locus 11543m5 wherein its 5′ flanking region comprises a nucleotide sequence of SEQ ID NO:9 and its 3′ flanking region comprises a nucleotide sequence complementary to SEQ ID NO:10;
(vi) a microsatellite locus 530m4 wherein its 5′ flanking region comprises a nucleotide sequence of SEQ ID NO:11 and its 3′ flanking region comprises a nucleotide sequence complementary to SEQ ID NO:12;
(vii) a microsatellite locus 11401m4 wherein its 5′ flanking region comprises a nucleotide sequence of SEQ ID NO:13 and its 3′ flanking region comprises a nucleotide sequence complementary to SEQ ID NO:14;
(viii) a microsatellite locus 441m6 wherein its 5′ flanking region comprises a nucleotide sequence of SEQ ID NO:15 and its 3′ flanking region comprises a nucleotide sequence complementary to SEQ ID NO:16;
(ix) a microsatellite locus 11292m4 wherein its 5′ flanking region comprises a nucleotide sequence of SEQ ID NO:17 and its 3′ flanking region comprises a nucleotide sequence complementary to SEQ ID NO:18;
(x) a microsatellite locus 8499m4 wherein its 5′ flanking region comprises a nucleotide sequence of SEQ ID NO:19 and its 3′ flanking region comprises a nucleotide sequence complementary to SEQ ID NO:20;
(xi) a microsatellite locus 7203m5 wherein its 5′ flanking region comprises a nucleotide sequence of SEQ ID NO:21 and its 3′ flanking region comprises a nucleotide sequence complementary to SEQ ID NO:22;
(xii) a microsatellite locus 10366m5 wherein its 5′ flanking region comprises a nucleotide sequence of SEQ ID NO:23 and its 3′ flanking region comprises a nucleotide sequence complementary to SEQ ID NO:24;
(xiii) a microsatellite locus 12130m5 wherein its 5′ flanking region comprises a nucleotide sequence of SEQ ID NO:25 and its 3′ flanking region comprises a nucleotide sequence complementary to SEQ ID NO:26;
(xiv) a microsatellite locus 4546m2 wherein its 5′ flanking region comprises a nucleotide sequence of SEQ ID NO:27 and its 3′ flanking region comprises a nucleotide sequence complementary to SEQ ID NO:28;
(xv) a microsatellite locus 9079m3 wherein its 5′ flanking region comprises a nucleotide sequence of SEQ ID NO:29 and its 3′ flanking region comprises a nucleotide sequence complementary to SEQ ID NO:30;
(xvi) a microsatellite locus 11192m4 wherein its 5′ flanking region comprises a nucleotide sequence of SEQ ID NO:31 and its 3′ flanking region comprises a nucleotide sequence complementary to SEQ ID NO:32;
(xvii) a microsatellite locus 8010m6 wherein its 5′ flanking region comprises a nucleotide sequence of SEQ ID NO:33 and its 3′ flanking region comprises a nucleotide sequence complementary to SEQ ID NO:34;
(xviii) a microsatellite locus 12198m3 wherein its 5′ flanking region comprises a nucleotide sequence of SEQ ID NO:35 and its 3′ flanking region comprises a nucleotide sequence complementary to SEQ ID NO:36; and
(xix) a microsatellite locus 7850m4 wherein its 5′ flanking region comprises a nucleotide sequence of SEQ ID NO:37 and its 3′ flanking region comprises a nucleotide sequence complementary to SEQ ID NO:38.

2. The method according to claim 1, wherein the detection of a microsatellite locus in the genome of the subject is performed by using one or more primer pairs selected from the group consisting of:
(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38.

3. The method of claim 2, wherein the one or more primer pairs are selected from the group consisting of:
(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer comprising a nucleotide sequence of SEQ ID NO:36; and
(xix) a primer comprising a nucleotide sequence of SEQ ID NO:37 and a primer comprising a nucleotide sequence of SEQ ID NO:38.

4. The method of claim 2,
wherein one of the primer pair further comprises a nucleotide sequence of SEQ ID NO: 39 (M13 primer) at its 5′ end.

5. A primer pair for amplifying a microsatellite locus in the genome of Acropora genus, wherein said microsatellite locus is selected from the group consisting of:
(i) a microsatellite locus 8346m3 wherein its 5′ flanking region comprises a nucleotide sequence of SEQ ID NO:1 and its 3′ flanking region comprises a nucleotide sequence complementary to SEQ ID NO:2;
(ii) a microsatellite locus 7961m4 wherein its 5′ flanking region comprises a nucleotide sequence of SEQ ID NO:3 and its 3′ flanking region comprises a nucleotide sequence complementary to SEQ ID NO:4;
(iii) a microsatellite locus 11745m3 wherein its 5′ flanking region comprises a nucleotide sequence of SEQ ID NO:5 and its 3′ flanking region comprises a nucleotide sequence complementary to SEQ ID NO:6;
(iv) a microsatellite locus 12406m3 wherein its 5′ flanking region comprises a nucleotide sequence of SEQ ID NO:7 and its 3′ flanking region comprises a nucleotide sequence complementary to SEQ ID NO:8;
(v) a microsatellite locus 11543m5 wherein its 5′ flanking region comprises a nucleotide sequence of SEQ ID NO:9 and its 3′ flanking region comprises a nucleotide sequence complementary to SEQ ID NO:10;
(vi) a microsatellite locus 530m4 wherein its 5′ flanking region comprises a nucleotide sequence of SEQ ID NO:11 and its 3′ flanking region comprises a nucleotide sequence complementary to SEQ ID NO:12;
(vii) a microsatellite locus 11401m4 wherein its 5′ flanking region comprises a nucleotide sequence of SEQ ID NO:13 and its 3′ flanking region comprises a nucleotide sequence complementary to SEQ ID NO:14;
(viii) a microsatellite locus 441m6 wherein its 5′ flanking region comprises a nucleotide sequence of SEQ ID NO:15 and its 3′ flanking region comprises a nucleotide sequence complementary to SEQ ID NO:16;
(ix) a microsatellite locus 11292m4 wherein its 5′ flanking region comprises a nucleotide sequence of SEQ ID NO:17 and its 3′ flanking region comprises a nucleotide sequence complementary to SEQ ID NO:18;
(x) a microsatellite locus 8499m4 wherein its 5′ flanking region comprises a nucleotide sequence of SEQ ID NO:19 and its 3′ flanking region comprises a nucleotide sequence complementary to SEQ ID NO:20;
(xi) a microsatellite locus 7203m5 wherein its 5′ flanking region comprises a nucleotide sequence of SEQ ID NO:21 and its 3′ flanking region comprises a nucleotide sequence complementary to SEQ ID NO:22;
(xii) a microsatellite locus 10366m5 wherein its 5′ flanking region comprises a nucleotide sequence of SEQ ID NO:23 and its 3′ flanking region comprises a nucleotide sequence complementary to SEQ ID NO:24;
(xiii) a microsatellite locus 12130m5 wherein its 5′ flanking region comprises a nucleotide sequence of SEQ ID NO:25 and its 3′ flanking region comprises a nucleotide sequence complementary to SEQ ID NO:26;
(xiv) a microsatellite locus 4546m2 wherein its 5′ flanking region comprises a nucleotide sequence of SEQ ID NO:27 and its 3′ flanking region comprises a nucleotide sequence complementary to SEQ ID NO:28;
(xv) a microsatellite locus 9079m3 wherein its 5′ flanking region comprises a nucleotide sequence of SEQ ID NO:29 and its 3′ flanking region comprises a nucleotide sequence complementary to SEQ ID NO:30;
(xvi) a microsatellite locus 11192m4 wherein its 5′ flanking region comprises a nucleotide sequence of SEQ ID NO:31 and its 3′ flanking region comprises a nucleotide sequence complementary to SEQ ID NO:32;
(xvii) a microsatellite locus 8010m6 wherein its 5′ flanking region comprises a nucleotide sequence of SEQ ID NO:33 and its 3′ flanking region comprises a nucleotide sequence complementary to SEQ ID NO:34;
(xviii) a microsatellite locus 12198m3 wherein its 5′ flanking region comprises a nucleotide sequence of SEQ ID NO:35 and its 3′ flanking region comprises a nucleotide sequence complementary to SEQ ID NO:36; and
(xix) a microsatellite locus 7850m4 wherein its 5′ flanking region comprises a nucleotide sequence of SEQ ID NO:37 and its 3′ flanking region comprises a nucleotide sequence complementary to SEQ ID NO:38.

6. The primer pair according to claim 5, wherein the primer pair is selected from the group consisting of:
(i) a primer comprising at least 17 consecutive nucleotide sequence having at least 90% sequence identity to a sequence of 5′ flanking region comprising a nucleotide sequence of SEQ ID NO:1 and a primer comprising at least 17 consecutive nucleotide sequence having at least 90% sequence identity to a sequence complementary to a sequence of 3′ flanking region comprises a nucleotide sequence complementary to SEQ ID NO:2;
(ii) a primer comprising at least 17 consecutive nucleotide sequence having at least 90% sequence identity to a sequence of 5′ flanking region comprising a nucleotide sequence of SEQ ID NO:3 and a primer comprising at least 17 consecutive nucleotide sequence having at least 90% sequence identity to a sequence complementary to a sequence of 3′ flanking region comprises a nucleotide sequence complementary to SEQ ID NO:4;
(iii) a primer comprising at least 17 consecutive nucleotide sequence having at least 90% sequence identity to a sequence of 5′ flanking region comprising a nucleotide sequence of SEQ ID NO:5 and a primer comprising at least 17 consecutive nucleotide sequence having at least 90% sequence identity to a sequence complementary to a sequence of 3′ flanking region comprises a nucleotide sequence complementary to SEQ ID NO:6;
(iv) a primer comprising at least 17 consecutive nucleotide sequence having at least 90% sequence identity to a sequence of 5′ flanking region comprising a nucleotide sequence of SEQ ID NO:7 and a primer comprising at least 17 consecutive nucleotide sequence having at least 90% sequence identity to a sequence complementary to a sequence of 3′ flanking region comprises a nucleotide sequence complementary to SEQ ID NO:8;
(v) a primer comprising at least 17 consecutive nucleotide sequence having at least 90% sequence identity to a sequence of 5′ flanking region comprising a nucleotide sequence of SEQ ID NO:9 and a primer comprising at least 17 consecutive nucleotide sequence having at least 90% sequence identity to a sequence complementary to a sequence of 3′ flanking region comprises a nucleotide sequence complementary to SEQ ID NO:10;
(vi) a primer comprising at least 17 consecutive nucleotide sequence having at least 90% sequence identity to a sequence of 5′ flanking region comprising a nucleotide sequence of SEQ ID NO:11 and a primer comprising at least 17 consecutive nucleotide sequence having at least 90% sequence identity to a sequence complementary to a sequence of 3′ flanking region comprises a nucleotide sequence complementary to SEQ ID NO:12;
(vii) a primer comprising at least 17 consecutive nucleotide sequence having at least 90% sequence identity to a sequence of 5′ flanking region comprising a nucleotide sequence of SEQ ID NO:13 and a primer comprising at least 17 consecutive nucleotide sequence having at least 90% sequence identity to a sequence complementary to a sequence of 3′ flanking region comprises a nucleotide sequence complementary to SEQ ID NO:14;
(viii) a primer comprising at least 17 consecutive nucleotide sequence having at least 90% sequence identity to a sequence of 5′ flanking region comprising a nucleotide sequence of SEQ ID NO:15 and a primer comprising at least 17 consecutive nucleotide sequence having at least 90% sequence identity to a sequence complementary to a sequence of 3′ flanking region comprises a nucleotide sequence complementary to SEQ ID NO:16;
(ix) a primer comprising at least 17 consecutive nucleotide sequence having at least 90% sequence identity to a sequence of 5′ flanking region comprising a nucleotide sequence of SEQ ID NO:17 and a primer comprising at least 17 consecutive nucleotide sequence having at least 90% sequence identity to a sequence complementary to a sequence of 3′ flanking region comprises a nucleotide sequence complementary to SEQ ID NO:18;
(x) a primer comprising at least 17 consecutive nucleotide sequence having at least 90% sequence identity to a sequence of 5′ flanking region comprising a nucleotide sequence of SEQ ID NO:19 and a primer comprising at least 17 consecutive nucleotide sequence having at least 90% sequence identity to a sequence complementary to a sequence of 3′ flanking region comprises a nucleotide sequence complementary to SEQ ID NO:20;
(xi) a primer comprising at least 17 consecutive nucleotide sequence having at least 90% sequence identity to a sequence of 5′ flanking region comprising a nucleotide sequence of SEQ ID NO:21 and a primer comprising at least 17 consecutive nucleotide sequence having at least 90% sequence identity to a sequence complementary to a sequence of 3′ flanking region comprises a nucleotide sequence complementary to SEQ ID NO:22;
(xii) a primer comprising at least 17 consecutive nucleotide sequence having at least 90% sequence identity to a sequence of 5′ flanking region comprising a nucleotide sequence of SEQ ID NO:23 and a primer comprising at least 17 consecutive nucleotide sequence having at least 90% sequence identity to a sequence complementary to a sequence of 3′ flanking region comprises a nucleotide sequence complementary to SEQ ID NO:24;
(xiii) a primer comprising at least 17 consecutive nucleotide sequence having at least 90% sequence identity to a sequence of 5′ flanking region comprising a nucleotide sequence of SEQ ID NO:25 and a primer comprising at least 17 consecutive nucleotide sequence having at least 90% sequence identity to a sequence complementary to a sequence of 3′ flanking region comprises a nucleotide sequence complementary to SEQ ID NO:26;
(xiv) a primer comprising at least 17 consecutive nucleotide sequence having at least 90% sequence identity to a sequence of 5′ flanking region comprising a nucleotide sequence of SEQ ID NO:27 and a primer comprising at least 17 consecutive nucleotide sequence having at least 90% sequence identity to a sequence complementary to a sequence of 3′ flanking region comprises a nucleotide sequence complementary to SEQ ID NO:28;
(xv) a primer comprising at least 17 consecutive nucleotide sequence having at least 90% sequence identity to a sequence of 5′ flanking region comprising a nucleotide sequence of SEQ ID NO:29 and a primer comprising at least 17 consecutive nucleotide sequence having at least 90% sequence identity to a sequence complementary to a sequence of 3′ flanking region comprises a nucleotide sequence complementary to SEQ ID NO:30;
(xvi) a primer comprising at least 17 consecutive nucleotide sequence having at least 90% sequence identity to a sequence of 5′ flanking region comprising a nucleotide sequence of SEQ ID NO:31 and a primer comprising at least 17 consecutive nucleotide sequence having at least 90% sequence identity to a sequence complementary to a sequence of 3′ flanking region comprises a nucleotide sequence complementary to SEQ ID NO:32;
(xvii) a primer comprising at least 17 consecutive nucleotide sequence having at least 90% sequence identity to a sequence of 5′ flanking region comprising a nucleotide sequence of SEQ ID NO:33 and a primer comprising at least 17 consecutive nucleotide sequence having at least 90% sequence identity to a sequence complementary to a sequence of 3′ flanking region comprises a nucleotide sequence complementary to SEQ ID NO:34;
(xviii) a primer comprising at least 17 consecutive nucleotide sequence having at least 90% sequence identity to a sequence of 5′ flanking region comprising a nucleotide sequence of SEQ ID NO:35 and a primer comprising at least 17 consecutive nucleotide sequence having at least 90% sequence identity to a sequence complementary to a sequence of 3′ flanking region comprises a nucleotide sequence complementary to SEQ ID NO:36; and
(xix) a primer comprising at least 17 consecutive nucleotide sequence having at least 90% sequence identity to a sequence of 5′ flanking region comprising a nucleotide sequence of SEQ ID NO:37 and a primer comprising at least 17 consecutive nucleotide sequence having at least 90% sequence identity to a sequence complementary to a sequence of 3′ flanking region comprises a nucleotide sequence complementary to SEQ ID NO:38.

7. The primer pair according to claim 6, wherein the primer pair is selected from the group consisting of:
(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26;
(xiv) a primer comprising a nucleotide sequence of SEQ ID NO:27 and a primer comprising a nucleotide sequence of SEQ ID NO:28;
(xv) a primer comprising a nucleotide sequence of SEQ ID NO:29 and a primer comprising a nucleotide sequence of SEQ ID NO:30;
(xvi) a primer comprising a nucleotide sequence of SEQ ID NO:31 and a primer comprising a nucleotide sequence of SEQ ID NO:32;
(xvii) a primer comprising a nucleotide sequence of SEQ ID NO:33 and a primer comprising a nucleotide sequence of SEQ ID NO:34;
(xviii) a primer comprising a nucleotide sequence of SEQ ID NO:35 and a primer comprising a nucleotide sequence of SEQ ID NO:36; and
(xix) a primer comprising a nucleotide sequence of SEQ ID NO:37 and a primer comprising a nucleotide sequence of SEQ ID NO:38.

8. The primer pair according to claim 5, wherein one of the primer pair further comprises a nucleotide sequence of SEQ ID NO: 39 (M13 primer) at its 5′ end.

9. A method for identifying an allele of a microsatellite locus which exhibits PCR size polymorphism in different genotypes of an Acropora species, comprising of:
identifying a microsatellite locus whose PCR fragment size, amplified with genomic DNA derived from a first coral colony of an Acropora species as template, is different from the PCR fragment size amplified with genomic DNA derived from a second coral colony of the Acropora species,
wherein said microsatellite locus is selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28.

10. A kit for determining a PCR size polymorphic allele of a microsatellite locus of an Acropora species, comprising
a primer pair which amplifies PCR fragment from the microsatellite locus, wherein said microsatellite locus is selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28.

11. The kit according to claim 10, wherein the primer pair is selected from the group consisting of:
(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28.

12. The kit according to claim 11, wherein the primer pair is selected from the group consisting of:
(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26; and
(xiv) a primer comprising a nucleotide sequence of SEQ ID NO:27 and a primer comprising a nucleotide sequence of SEQ ID NO:28.

13. A method for quantifying an index of level of chimerism of a coral colony restored by transplanting a first coral colony of an Acropora species or a part thereof with a second coral colony in need of restoration that belongs to the same species as the first coral colony, comprising of:
(a) identifying one or more microsatellite loci whose PCR fragment size, amplified with genomic DNA derived from the first coral colony as template, is different from the PCR fragment size amplified with genomic DNA derived from the second coral colony,
wherein said microsatellite loci are selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28;
(b) determining the molar percentages of the genomic DNA for the alleles representing the first and second coral colonies, and
(c) providing the molar percentage of the PCR fragment representing the first coral colony as the index of level of chimerism of the chimeric coral colony restored.

14. A kit for quantifying an index of level of chimerism of a coral colony restored by transplanting a first coral colony of an Acropora species or a part thereof with a second coral colony in need of restoration that belongs to the same species as the first coral colony, comprising
a primer pair which amplify a microsatellite locus in the genome of the subject, wherein said microsatellite loci are selected from the group consisting of:
(i) a microsatellite locus 8346m3 having a 5′ flanking region that comprises a nucleotide sequence of SEQ ID NO:1 and a 3′ flanking region that comprises a nucleotide sequence complementary to SEQ ID NO:2;
(ii) a microsatellite locus 7961m4 having a 5′ flanking region that comprises a nucleotide sequence of SEQ ID NO:3 and a 3′ flanking region that comprises a nucleotide sequence complementary to SEQ ID NO:4;
(iii) a microsatellite locus 11745m3 having a 5′ flanking region that comprises a nucleotide sequence of SEQ ID NO:5 and a 3′ flanking region that comprises a nucleotide sequence complementary to SEQ ID NO:6;
(iv) a microsatellite locus 12406m3 having a 5′ flanking region that comprises a nucleotide sequence of SEQ ID NO:7 and a 3′ flanking region that comprises a nucleotide sequence complementary to SEQ ID NO:8;
(v) a microsatellite locus 11543m5 having a 5′ flanking region that comprises a nucleotide sequence of SEQ ID NO:9 and a 3′ flanking region that comprises a nucleotide sequence complementary to SEQ ID NO:10;
(vi) a microsatellite locus 530m4 having a 5′ flanking region that comprises a nucleotide sequence of SEQ ID NO:11 and a 3′ flanking region that comprises a nucleotide sequence complementary to SEQ ID NO:12;
(vii) a microsatellite locus 11401m4 having a 5′ flanking region that comprises a nucleotide sequence of SEQ ID NO:13 and a 3′ flanking region that comprises a nucleotide sequence complementary to SEQ ID NO:14;
(viii) a microsatellite locus 441m6 having a 5′ flanking region that comprises a nucleotide sequence of SEQ ID NO:15 and a 3′ flanking region that comprises a nucleotide sequence complementary to SEQ ID NO:16;
(ix) a microsatellite locus 11292m4 having a 5′ flanking region that comprises a nucleotide sequence of SEQ ID NO:17 and a 3′ flanking region that comprises a nucleotide sequence complementary to SEQ ID NO:18;
(x) a microsatellite locus 8499m4 having a 5′ flanking region that comprises a nucleotide sequence of SEQ ID NO:19 and a 3′ flanking region that comprises a nucleotide sequence complementary to SEQ ID NO:20;
(xi) a microsatellite locus 7203m5 having a 5′ flanking region that comprises a nucleotide sequence of SEQ ID NO:21 and a 3′ flanking region that comprises a nucleotide sequence complementary to SEQ ID NO:22;
(xii) a microsatellite locus 10366m5 having a 5′ flanking region that comprises a nucleotide sequence of SEQ ID NO:23 and a 3′ flanking region that comprises a nucleotide sequence complementary to SEQ ID NO:24;
(xiii) a microsatellite locus 12130m5 having a 5′ flanking region that comprises a nucleotide sequence of SEQ ID NO:25 and a 3′ flanking region that comprises a nucleotide sequence complementary to SEQ ID NO:26; and
(xiv) a microsatellite locus 4546m2 having a 5′ flanking region that comprises a nucleotide sequence of SEQ ID NO:27 and a 3′ flanking region that comprises a nucleotide sequence complementary to SEQ ID NO:28.

15. The kit according to claim 14, wherein the primer pair is selected from the group consisting of:
(i) a primer comprising at least 17 consecutive nucleotides having at least 90% sequence identity to a nucleotide sequence of SEQ ID NO:1 and a primer comprising at least 17 consecutive nucleotides having at least 90% sequence identity to a nucleotide sequence complementary to SEQ ID NO:2;
(ii) a primer comprising at least 17 consecutive nucleotides having at least 90% sequence identity to a nucleotide sequence of SEQ ID NO:3 and a primer comprising at least 17 consecutive nucleotides having at least 90% sequence identity to a nucleotide sequence complementary to SEQ ID NO:4;
(iii) a primer comprising at least 17 consecutive nucleotides having at least 90% sequence identity to a nucleotide sequence of SEQ ID NO:5 and a primer comprising at least 17 consecutive nucleotides having at least 90% sequence identity to a nucleotide sequence complementary to SEQ ID NO:6;
(iv) a primer comprising at least 17 consecutive nucleotides having at least 90% sequence identity to a nucleotide sequence of SEQ ID NO:7 and a primer comprising at least 17 consecutive nucleotides having at least 90% sequence identity to a nucleotide sequence complementary to SEQ ID NO:8;
(v) a primer comprising at least 17 consecutive nucleotides having at least 90% sequence identity to a nucleotide sequence of SEQ ID NO:9 and a primer comprising at least 17 consecutive nucleotides having at least 90% sequence identity to a nucleotide sequence complementary to SEQ ID NO:10;
(vi) a primer comprising at least 17 consecutive nucleotides having at least 90% sequence identity to a nucleotide sequence of SEQ ID NO:11 and a primer comprising at least 17 consecutive nucleotides having at least 90% sequence identity to a nucleotide sequence complementary to SEQ ID NO:12;
(vii) a primer comprising at least 17 consecutive nucleotides having at least 90% sequence identity to a nucleotide sequence of SEQ ID NO:13 and a primer comprising at least 17 consecutive nucleotides having at least 90% sequence identity to a nucleotide sequence complementary to SEQ ID NO:14;
(viii) a primer comprising at least 17 consecutive nucleotides having at least 90% sequence identity to a nucleotide sequence of SEQ ID NO:15 and a primer comprising at least 17 consecutive nucleotides having at least 90% sequence identity to a nucleotide sequence complementary to SEQ ID NO:16;
(ix) a primer comprising at least 17 consecutive nucleotides having at least 90% sequence identity to a nucleotide sequence of SEQ ID NO:17 and a primer comprising at least 17 consecutive nucleotides having at least 90% sequence identity to a nucleotide sequence complementary to SEQ ID NO:18;
(x) a primer comprising at least 17 consecutive nucleotides having at least 90% sequence identity to a nucleotide sequence of SEQ ID NO:19 and a primer comprising at least 17 consecutive nucleotides having at least 90% sequence identity to a nucleotide sequence complementary to SEQ ID NO:20;
(xi) a primer comprising at least 17 consecutive nucleotides having at least 90% sequence identity to a nucleotide sequence of SEQ ID NO:21 and a primer comprising at least 17 consecutive nucleotides having at least 90% sequence identity to a nucleotide sequence complementary to SEQ ID NO:22;
(xii) a primer comprising at least 17 consecutive nucleotides having at least 90% sequence identity to a nucleotide sequence of SEQ ID NO:23 and a primer comprising at least 17 consecutive nucleotides having at least 90% sequence identity to a nucleotide sequence complementary to SEQ ID NO:24;
(xiii) a primer comprising at least 17 consecutive nucleotides having at least 90% sequence identity to a nucleotide sequence of SEQ ID NO:25 and a primer comprising at least 17 consecutive nucleotides having at least 90% sequence identity to a nucleotide sequence complementary to SEQ ID NO:26; and
(xiv) a primer comprising at least 17 consecutive nucleotides having at least 90% sequence identity to a nucleotide sequence of SEQ ID NO:27 and a primer comprising at least 17 consecutive nucleotides having at least 90% sequence identity to a nucleotide sequence complementary to SEQ ID NO:28.

16. The kit according to claim 15, wherein the primer pair is selected from the group consisting of:
(i) a primer comprising a nucleotide sequence of SEQ ID NO:1 and a primer comprising a nucleotide sequence of SEQ ID NO:2;
(ii) a primer comprising a nucleotide sequence of SEQ ID NO:3 and a primer comprising a nucleotide sequence of SEQ ID NO:4;
(iii) a primer comprising a nucleotide sequence of SEQ ID NO:5 and a primer comprising a nucleotide sequence of SEQ ID NO:6;
(iv) a primer comprising a nucleotide sequence of SEQ ID NO:7 and a primer comprising a nucleotide sequence of SEQ ID NO:8;
(v) a primer comprising a nucleotide sequence of SEQ ID NO:9 and a primer comprising a nucleotide sequence of SEQ ID NO:10;
(vi) a primer comprising a nucleotide sequence of SEQ ID NO:11 and a primer comprising a nucleotide sequence of SEQ ID NO:12;
(vii) a primer comprising a nucleotide sequence of SEQ ID NO:13 and a primer comprising a nucleotide sequence of SEQ ID NO:14;
(viii) a primer comprising a nucleotide sequence of SEQ ID NO:15 and a primer comprising a nucleotide sequence of SEQ ID NO:16;
(ix) a primer comprising a nucleotide sequence of SEQ ID NO:17 and a primer comprising a nucleotide sequence of SEQ ID NO:18;
(x) a primer comprising a nucleotide sequence of SEQ ID NO:19 and a primer comprising a nucleotide sequence of SEQ ID NO:20;
(xi) a primer comprising a nucleotide sequence of SEQ ID NO:21 and a primer comprising a nucleotide sequence of SEQ ID NO:22;
(xii) a primer comprising a nucleotide sequence of SEQ ID NO:23 and a primer comprising a nucleotide sequence of SEQ ID NO:24;
(xiii) a primer comprising a nucleotide sequence of SEQ ID NO:25 and a primer comprising a nucleotide sequence of SEQ ID NO:26; and
(xiv) a primer comprising a nucleotide sequence of SEQ ID NO:27 and a primer comprising a nucleotide sequence of SEQ ID NO:28.

17. The method of claim 3, wherein one of the primer pair further comprises a nucleotide sequence of SEQ ID NO: 39 (M13 primer) at its 5′ end.

18. The primer pair according to claim 6, wherein one of the primer pair further comprises a nucleotide sequence of SEQ ID NO: 39 (M13 primer) at its 5′ end.

19. The primer pair according to claim 7, wherein one of the primer pair further comprises a nucleotide sequence of SEQ ID NO: 39 (M13 primer) at its 5′ end.
